(19) United States
(12) Patent Application Publication
(10) Pub. No.: US 2024/0197257 A1
(43) Pub. Date: Jun. 20, 2024

(54) Healthy Selfie[TM]: Methods and Devices for Using a Cellphone for Guided Capture of Images of a Patient's Body from Different Angles and Distances

(71) Applicant: Robert A. Connor, Wyoming, MN (US)

(72) Inventor: Robert A. Connor, Wyoming, MN (US)

(73) Assignee: Holovisions LLC, Ham Lake, MN (US)

(21) Appl. No.: 18/431,946

(22) Filed: Feb. 3, 2024

Related U.S. Application Data
(63) Continuation-in-part of application No. 17/722,979, filed on Apr. 18, 2022.
Application No. 17/722,979 is a continuation-in-part of application No. 17/404,174, filed on Aug. 17, 2021, now Pat. No. 11,308,618.
Application No. 17/404,174 is a continuation-in-part of application No. 16/706,111, filed on Dec. 6, 2019, now Pat. No. 11,176,669.
(60) Provisional application No. 62/833,761, filed on Apr. 14, 2019.

(51) Int. Cl. A61B 5/00; B25J 9/12; G16H 40/63

(52) U.S. Cl. CPC A61B 5/6898; A61B 5/0013; A61B 5/0077; A61B 5/7267; A61B 5/741; A61B 5/745; B25J 9/12; G16H 40/63; A61B 2576/00

(57) ABSTRACT

Disclosed herein are methods and devices for using a cellphone to capture images of a portion of a patient's body from different angles and distances. These images are integrated together to create a digital 3D image of the portion of the person's body which is: analyzed using machine learning and/or artificial intelligence; and/or examined by a remote healthcare provider.

### CROSS-REFERENCE TO RELATED APPLICATIONS

This application is a continuation-in-part of U.S. patent application Ser. No. 17/722,979 which was filed on 2022 Apr. 18. U.S. patent application Ser. No. 17/722,979 was a continuation-in-part of U.S. patent application Ser. No. 17/404,174 which was filed on 2021 Aug. 17 and issued as U.S. patent Ser. No. 11/308,618 on 2022 Apr. 19. U.S. patent application Ser. No. 17/404,174 was a continuation-in-part of U.S. patent application Ser. No. 16/706,111 which was filed on 2019 Dec. 6 and issued as U.S. patent Ser. No. 11/176,669 on 2021 Nov. 16. U.S. patent application Ser. No. 16/706,111 claimed the priority benefit of U.S. provisional patent application 62/833,761 filed on 2019 Apr. 14. The entire contents of these related applications are incorporated herein by reference.

### FEDERALLY SPONSORED RESEARCH

Not Applicable

### SEQUENCE LISTING OR PROGRAM

### Not Applicable

### BACKGROUND

### Field of Invention

This invention relates to remote medical imaging using conventional mobile devices.

### Introduction

There are many reasons for remote medical evaluation via electronic communication (e.g. telemedicine). With remote medical evaluation, people in low-population areas who do not have local healthcare specialists can have remote access to care from non-local specialists. Also, remote medical evaluation can help people with limited transportation to receive care from their homes and help people with busy work schedules get care at convenient times. Further, as highlighted by the recent pandemic, remote medical evaluation can help people to avoid the in-person contact which can spread contagious disease. Also, from a provider perspective, remote medical evaluation can enhance provider productivity, lower the cost of care, and broaden a provider's geographic service area.

For these reasons, as well as technological advances, there has been progress in remote medical evaluation during the past several years. The quality and functionality of internet-based medical video conferencing has improved. Mobile phones have become ubiquitous and the quality of images captured by their cameras has improved. Increasingly-sophisticated software, machine-learning, and AI programs are playing a greater role in telemedicine and virtual care portals. Accordingly, remote medical evaluation is growing.

Despite these advances, however, there are still significant challenges remote medical evaluation, especially for remote imaging. Potential problems include: improper distance from the phone to the body, out-of-focus image, and insufficient cues to assess the size and orientation of an area of interest; improper angle or limited number of angles between the phone and the body which make it difficult to assess three-dimensional attributes of the area; improper ambient lighting level or color, and differences in image color calibration, caused by different types of phones, which confound provider assessment of body color.

As will be discussed in the next section, some innovative companies have begun to tackle these problems with remote medical imaging for telemedicine. Some are creating customized medical devices, such as medical robots, which are used in healthcare facilities. However, thus far these customized medical devices tend to be expensive and not suited for home use. Remote medical evaluation would be greatly facilitated by a relatively low-cost technology which takes full advantage of the wide-spread use of conventional mobile devices. Some innovative companies are developing products which take advantage of conventional mobile phones for medical purposes. Their products include fiducial strips and stickers (with size markings and colors) which are placed near a person's body to improve calibration and assessment of the size, shape, and/or color of a body area in a phone-based image. Such strips and sticks are also being used as a color reference to which a color-changing medical test strip can be compared. Other innovative companies are focusing on software, machine learning, three-dimensional models, and tracking changes in a body area over time.

However, despite these innovations, there is an unmet need for portable, low-cost, and easy-to-use technology to guide remote medical imaging using conventional camera-equipped mobile phones to address the medical imaging problems noted above, especially those related to distance, angle, lighting, and color. This need is addressed by the invention (the “Healthy Selfie”™) which is disclosed herein.

### REVIEW OF THE RELEVANT ART

This review of the relevant art starts with a review of patents and patent applications. U.S. Pat. No. 5,016,173 (Kenet et al., May 14, 1991, “Apparatus and Method for Monitoring Visually Accessible Surfaces of the Body”) discloses an apparatus and method for in vivo monitoring of body surfaces. U.S. patent application 20030085908 (Luby, May 8, 2003, “Method and Apparatus for an Automated Reference Indicator System for Photographic and Video Images”) and U.S. Pat. No. 6,873,340 (Luby, Mar. 29, 2005, “Method and Apparatus for an Automated Reference Indicator System for Photographic and Video Images”) disclose an automated reference indicator system with at least one indicator patch placed near an area of interest. U.S. patent application 20040019269 (Schaefer et al., Jan. 29, 2004, “Early Detection of Inflammation and Infection Using Infrared Thermography”) discloses a method for detecting inflammation using infrared thermography.

U.S. patent application 20040059199 (Thomas et al., Mar. 25, 2004, “Wound Assessment and Monitoring Apparatus and Method”) discloses a wound assessment and monitoring apparatus and method for monitoring the development of wounds by patients of a health care facility. U.S. patent application 20040220464 (Benninger et al., Nov. 4, 2004, “Method and Apparatus for Carrying Out a Televisit”) discloses a method of carrying out a televisit comprising acquiring the data of a patient, recording an image of at least one body zone of the patient, and transmitting the data and the image to a medical institution.

U.S. Pat. No. 6,925,357 (Wang et al., Aug. 2, 2005, “Medical Tele-Robotic System”), U.S. Pat. No. 7,142,945 (Wang et al., Nov. 28, 2006, “Medical Tele-Robotic System”), U.S. Pat. No. 7,142,947 (Wang et al., Nov. 28, 2006, “Medical Tele-Robotic Method”), U.S. Pat. No. 7,164,970 (Wang et al., Jan. 16, 2007, “Medical Tele-Robotic System”), and U.S. Pat. No. 7,218,992 (Wang et al., May 15, 2007, “Medical Tele-Robotic System”) disclose a remote-controlled telemedicine robot. U.S. Pat. No. 7,158,860 (Wang et al., Jan. 2, 2007, “Healthcare Tele-Robotic System Which Allows Parallel Remote Station Observation”) and U.S. Pat. No. 7,171,286 (Wang et al., Jan. 30, 2007, “Healthcare Tele-Robotic System with a Robot That Also Functions as a Remote Station”) disclose a mobile medical robot which provides audio and visual information. U.S. Pat. No. 7,161,322 (Wang et al., Jan. 9, 2007, “Robot with a Manipulator Arm”) discloses a robot with an arm coupled to a platform.

U.S. patent application 20070112464 (Wang et al., May 17, 2007, “Apparatus and Method for Patient Rounding with a Remote Controlled Robot”) and U.S. Pat. No. 7,164,969 (Wang et al., Jan. 16, 2007. “Apparatus and Method for Patient Rounding with a Remote Controlled Robot”) disclose a remote-controlled medical robot. U.S. patent applications 20120197439 (Wang et al., Aug. 2, 2012, “Interfacing with a Mobile Telepresence Robot”) and U.S. patent application 20120197464 (Wang et al., Aug. 2, 2012. “Interfacing with a Mobile Telepresence Robot”) and U.S. Pat. No. 8,718,837 (Wang et al., May 6, 2014, “Interfacing with a Mobile Telepresence Robot”), U.S. Pat. No. 8,965,579 (Wang et al., Feb. 24, 2015, “Interfacing with a Mobile Telepresence Robot”), and U.S. Pat. No. 9,469,030 (Wang et al., Oct. 18, 2016, “Interfacing with a Mobile Telepresence Robot”) disclose a mobile telepresence robot with a drive system, a control system, an imaging system, and a mapping module.

U.S. Pat. No. 7,162,063 (Craine et al., Jan. 9, 2007, “Digital Skin Lesion Imaging System and Method”) discloses detection of skin lesions using digital baseline image data of an area, a physical calibration piece near the area, and monitoring area changes. U.S. patent application 20090093688 (Mathur, Apr. 9, 2009, “System, Device, and Method for Remote Monitoring and Servicing”) discloses an interface device with a built-in video camera which is configured to send video information from the built-in video camera as well as physiological information to a remote monitoring facility.

U.S. patent applications 20100091104 (Sprigle et al., Apr. 15, 2010, “Systems and Methods for the Measurement of Surfaces”) and 20120035469 (Whelan et al., Feb. 9, 2012, “Systems and Methods for the Measurement of Surfaces”) disclose a portable, hand-held, non-contact surface measuring system comprising an image capturing element, at least four projectable reference elements positioned parallel to one another at known locations around the image capturing element, a processing unit, and a user interface. U.S. patent application 20100271470 (Stephan et al., Oct. 28, 2010, “Method and Apparatus for Characterizing a Person's Skin Imperfections”) discloses a method of characterizing a person's skin imperfections using a digital color image-taking device.

U.S. patent application 20110013006 (Uzenbajakava et al., Jan. 20, 2011, “Apparatus for Skin Imaging. System for Skin Analysis”) discloses an apparatus for skin imaging which captures near-field and far-field skin images under different illumination angles. U.S. patent application 20110216204 (Elwell et al., Sep. 8, 2011, “Systems and Methods for Bio-Image Calibration”) discloses products for bio-image calibration.

U.S. patent application 20120218379 (Ozcan et al., Aug. 30, 2012, “Incoherent Lensfree Cell Holography and Microscopy on a Chip”) and U.S. Pat. No. 9,007,433 (Ozcan et al., Apr. 14, 2015, “Incoherent Lensfree Cell Holography and Microscopy on a Chip”) disclose a system for imaging a cytological sample including a sample holder which holds a cytological sample. U.S. patent application 20120259229 (Wang et al., Oct. 11, 2012. “Apparatus and Methods for In Vivo Tissue Characterization by Raman Spectroscopy”) discloses a spectrometer system for differentiating tumor lesions. U.S. patent application 20130053677 (Schoenfeld, Feb. 28, 2013, “System and Method for Wound Care Management Based on a Three Dimensional Image of a Foot”) discloses a system for wound care management using a scanner configured to obtain a two-dimensional image of the plantar surface of the foot.

U.S. patent application 20130128223 (Wood et al., May 23, 2013, “Digital-Based Medical Devices”) discloses a hand-held ophthalmic examination instrument which emits amber light and white light. U.S. patent application 20130331708 (Estocado et al., Dec. 12, 2013, “Diagnostic Imaging System for Skin and Affliction Assessment”) and U.S. Pat. No. 9,161,716 (Estocado, Oct. 20, 2015, “Diagnostic Imaging System for Skin and Affliction Assessment”) disclose a size-assessment tool which is placed near an affliction for imaging. U.S. Pat. No. 8,638,986 (Jiang et al., Jan. 28, 2014. “Online Reference Patch Generation and Pose Estimation for Augmented Reality”) discloses a reference patch generated using a captured image of a planar object with two perpendicular sets of parallel lines. U.S. patent application 20140029815 (Kadir et al., Jan. 30, 2014, “Measurement System for Medical Images”) discloses a method of measuring a parameter of a structure on a medical image with a measurement tool displayed on a slice of the image.

U.S. Pat. No. 8,755,053 (Fright et al., Jun. 17, 2014, “Method of Monitoring a Surface Feature and Apparatus Therefor”), U.S. Pat. No. 9,377,295 (Fright et al., Jun. 28, 2016, “Method of Monitoring a Surface Feature and Apparatus Therefor”), and U.S. Pat. No. 9,955,910 (Fright et al., May 1, 2018, “Method of Monitoring a Surface Feature and Apparatus Therefor”) disclose determination of the dimensions of a surface feature by capturing an image of the surface feature and determining a scale associated with the image. U.S. patent application 20170000351 (Fright et al., Jan. 5, 2017, “Handheld Skin Measuring or Monitoring Device”) and U.S. Pat. No. 9,179,844 (Fright et al., Nov. 10, 2015, “Handheld Skin Measuring or Monitoring Device”) and U.S. Pat. No. 9,861,285 (Fright et al., Jan. 9, 2018, “Handheld Skin Measuring or Monitoring Device”) disclose a handheld skin monitoring or measuring device with a camera and a structured light arrangement configured to project laser beams.

U.S. patent applications 20140139616 (Pinter et al., May 22, 2014, “Enhanced Diagnostics for a Telepresence Robot”), U.S. patent application 20140155755 (Pinter et al., Jun. 5, 2014, “Enhanced Diagnostics for a Telepresence Robot”), and U.S. patent application 20180263703 (Pinter et al., Sep. 30, 2018, “Enhanced Diagnostics for a Telepresence Robot”), and U.S. Pat. No. 9,974,612 (Pinter et al., May 22, 2018, “Enhanced Diagnostics for a Telepresence Robot”) disclose a telepresence robot which may include an image sensor, a thermal camera, a depth sensor, and one or more systems for interacting with patients. U.S. patent application 20140257058 (Clarysse et al., Sep. 11, 2014, “Automated Personal Medical Diagnostic System, Method, and Arrangement”) discloses an automated personal medical diagnostic system with at least one sensor to measure at least one physiological condition.

U.S. patent application 20140300722 (Garcia, Oct. 9, 2014, “Image-Based Measurement Tools”) and U.S. Pat. No. 9,696,897 (Garcia, Jul. 4, 2017, “Image-Based Measurement Tools,”) disclose using an available object (such as a coin, bank note, person of known height, sticker of known dimensions, printed object of known dimensions, or shape made of projected light) as a fiducial marker in mobile device imaging. U.S. patent application 20140313303 (Davis et al., Oct. 23, 2014, “Longitudinal Dermoscopic Study Employing Smartphone-Based Image Registration”) discloses how a user can capture skin images using a smartphone and these images can be co-registered, color-corrected, and presented to the user (or a clinician) for review.

U.S. patent application 20150002606 (Hyde et al., Jan. 1, 2015, “Medical Support System Including Medical Equipment Case”) discloses a medical equipment case for containing and transporting at least one article of medical equipment and a two-way audio-visual system. U.S. patent application 20150119652 (Hyde et al., Apr. 30, 2015, “Telemedicine Visual Monitoring Device with Structured Illumination”) discloses a system for providing telemedicine support with structured illumination and two-way audio visual communication between a patient and a remote caregiver. U.S. patent applications 20150278431 (Hyde et al., Oct. 1, 2015, “Quantified-Self Machines and Circuits Reflexively Related to Kiosk Systems and Associated Fabrication Machines and Circuits”) and 20150278480 (Hyde et al., Oct. 1, 2015, “Quantified-Self Machines and Circuits Reflexively Related to Kiosk Systems and Associated Food-And-Nutrition Machines and Circuits”) disclose a system and method electronically receives user biological status information from electronically involved detection of one or more biological user conditions.

U.S. patent application 20150036043 (Markovic et al., Feb. 5, 2015, “Bioscicon's Cellphone Camera—Microscope Universal Adapter”) discloses a device which simulates movement of a human examiner analyzing cytopathological specimens using a microscope. U.S. patent applications 20150044098 (Smart et al., Feb. 12, 2015, “Hyperspectral Imaging Systems, Units, and Methods”) and 20190281204 (Darty et al., Sep. 12, 2019, “Hyperspectral Imager Coupled with Indicator Molecule Tracking”) disclose methods and systems for concurrent imaging at multiple wavelengths. U.S. patent applications 20150119721 (Pedersen et al, Apr. 30, 2015, “System and Method for Assessing Wound”), 20180330522 (Pedersen et al., Nov. 15, 2018, “System and Method for Assessing Wound”), and 20180357763 (Pedersen et al., Dec. 13, 2018, “System and Method for Assessing Wound”) disclose a wound assessing method and system.

U.S. Pat. No. 9,042,967 (Dacosta et al., May 26, 2015, “Device and Method for Wound Imaging and Monitoring”) discloses a device for fluorescence-based imaging. U.S. patent applications 20150150457 (Wu et al., Jun. 4, 2015, “Method and System for Wound Assessment and Management”) and U.S. patent application 20160206205 (Wu et al., Jul. 21, 2016, “Method and System for Wound Assessment and Management”) disclose a system and method for determining characteristics of a wound with a first imaging sensor that obtains imaging information of a wound area and a second imaging sensor that obtains topology information of the wound area.

U.S. patent applications 20150308961 (Burg et al., Oct. 29, 2015, “Quantifying Color Changes of Chemical Test Pads Induced by Specific Concentrations of Biological Analytes Under Different Lighting Conditions”) and 20190310203 (Burg et al., Oct. 10, 2019, “Quantifying Color Changes of Chemical Test Pads Induced by Specific Concentrations of Biological Analytes Under Different Lighting Conditions”) and U.S. Pat. No. 9,285,323 (Burg et al., Mar. 15, 2016, “Quantifying Color Changes of Chemical Test Pads Induced Concentrations of Biological Analytes Under Different Lighting Conditions”) and (Burg et al., Apr. 23, 2019, “Quantifying Color Changes of Chemical Test Pads Induced by Specific Concentrations of Biological Analytes Under Different Lighting Conditions”) disclose color quantification of chemical test pads and titration of analytes under different lighting conditions.

U.S. patent applications 20150313484 (Burg et al., Nov. 5, 2015, “Portable Device with Multiple Integrated Sensors for Vital Signs Scanning”) and 20190298183 (Burg et al., Oct. 3, 2019, “Portable Device With Multiple Integrated Sensors for Vital Signs Scanning”) disclose a portable device with multiple integrated sensors for scanning vital signs. U.S. Pat. No. 9,607,380 (Burg et al., Mar. 28, 2017, “Methods and Apparatus for Quantifying Color Changes Induced by Specific Concentrations of Biological Analytes”) discloses methods and electronic devices for performing color-based reaction testing of biological materials.

U.S. patent applications 20170098137 (Burg et al., Apr. 6, 2017, “Method, Apparatus and System for Detecting and Determining Compromised Reagent Pads by Quantifying Color Changes Induced by Exposure to a Hostile Environment”) and 20200225166 (Burg et al., Jul. 16, 2020, “Method, Apparatus and System for Detecting and Determining Compromised Reagent Pads by Quantifying Color Changes Induced by Exposure to a Hostile Environment”) disclose reagent test paddle with a contamination detection medium, a reference color bar, at least one chemical test medium, and a unique identifier. U.S. patent application 20180252585 (Burg et al., Sep. 6, 2018, “Precision Luxmeter Methods for Digital Cameras to Quantify Colors in Uncontrolled Lighting Environments”) and U.S. Pat. No. 9,863,811 (Burg, Jan. 9, 2018, “Precision Luxmeter Methods for Digital Cameras to Quantify Colors in Uncontrolled Lighting Environments”) and 10948352 (Burg et al., Mar. 16, 2021, “Precision Luxmeter Methods for Digital Cameras to Quantify Colors in Uncontrolled Lighting Environments”) disclose a diagnostic system for biological samples including a diagnostic instrument and a portable electronic device.

U.S. patent application 20150359458 (Erickson et al., Dec. 17, 2015, “Smartphone-Based Apparatus and Method for Obtaining Repeatable, Quantitative Colorimetric Measurement”) discloses a method for obtaining a point-of-collection, selected quantitative indicia of an analyte on a test strip using a smartphone. U.S. patent application 20150379735 (Lim et al., Dec. 31, 2015, “Remote Monitoring Framework”) discloses a technology for facilitating remote monitoring using color data of a region of interest received by a computer system from a mobile device. U.S. patent application 20160042513 (Yudovsky et al., Feb. 11, 2016, “Systems and Methods for Evaluating Hyperspectral Imaging Data Using a Two Layer Media Model of Human Tissue”) discloses a method for acquiring a hyperspectral imaging data set from a region of interest of a human subject using a hyperspectral imager. U.S. patent application 20160163028 (Xu et al., Jun. 9, 2016, “Method and Device for Image Processing”) discloses a method comprising performing facial recognition on an image, determining a skin area to be processed, determining the locations of skin blemishes, and removing the skin blemishes in the image.

U.S. patent applications 20160248994 (Liu, Aug. 25, 2016, “Multipurpose Imaging and Display System”) and 20200053298 (Liu et al., Feb. 13, 2020, “Multipurpose Imaging and Display System”) disclose a multi-purpose imaging and display system including a display, a detector coupled to the display and having a field of view, and a filter communicating with the detector. U.S. patent application 20170053073 (Allen et al., Feb. 23, 2017, “System and Methods for Implementing Wound Therapy Protocols”) discloses systems, methods, and apparatuses for treating a tissue site. U.S. patent applications 20170099449 (Kang et al., Apr. 6, 2017, “Electronic Device and Method for Generating Image Data”), 20190141271 (Kang et al., May 9, 2019, “Electronic Device and Method for Generating Image Data”), and 20210203871 (Kang et al., Jul. 1, 2021, “Electronic Device and Method for Generating Image Data”) disclose an electronic device with an image sensor that acquires an optical signal corresponding to an object and a controller that controls the image sensor.

U.S. patent application 20170118404 (Song et al., Apr. 27, 2017, “Method for Setting Focus and Electronic Device Thereof”) discloses a method and apparatus for dynamically determining an auto focusing (AF) area according to a size information of a face in a digital image processing device. U.S. Pat. No. 9,674,407 (Gupta et al., Jun. 6, 2017, “System and Method for Interactive Image Capture for a Device Having a Camera”) discloses a guidance system for interactive image capture for use with a device having a camera and a camera lens. U.S. patent applications 20170229149 (Rothschild et al., Aug. 10, 2017, “System and Method for Using, Biometric, and Displaying Biometric Data”), 20190325914 (Rothschild et al., Oct. 24, 2019, “System and Method for Using, Processing, and Displaying Biometric Data”), and 20200126593 (Rothschild et al., Apr. 23, 2020, “System and Method for Using, Processing, and Displaying Biometric Data”) disclose a method for processing and displaying biometric data of a user.

U.S. patent application 20170293297 (Kim et al., Oct. 12, 2017, “Electronic Apparatus and Operating Method Thereof”) discloses a drone with a camera. U.S. patent application 20170315108 (Yoon et al., Nov. 2, 2017, “Rapid and Non-Destructive Detection of Infection”) discloses methods and devices to identify an infection via light scatter from a tissue surface. U.S. patent applications 20170316155 (Fairbairn et al., Nov. 2, 2017, “Automatically Assessing an Anatomical Surface Feature and Securely Managing Information Related to the Same”) and 20210225492 (Fairbairn et al., Jul. 22, 2021, “Automatically Assessing an Anatomical Surface Feature and Securely Managing Information Related to the Same”) disclose a facility for procuring information and including image information about an anatomical surface feature.

U.S. patent applications 20170316582 (Chen, Nov. 2, 2017, “Robust Head Pose Estimation with a Depth Camera”), 20170345183 (Chen, Nov. 30, 2017, “Robust Head Pose Estimation with a Depth Camera”), and 20200105013 (Chen, Apr. 2, 2020, “Robust Head Pose Estimation with a Depth Camera”) and U.S. patent Ser. No. 10/157,477 (Chen, Dec. 18, 2018, “Robust Head Pose Estimation with a Depth Camera”) and 10755438 (Chen, Aug. 25, 2020, “Robust Head Pose Estimation with a Depth Camera”) disclose systems and methods to estimate the pose of a human subject's head from a sequence of images received from a single depth camera.

U.S. Pat. No. 9,818,193 (Smart, Nov. 14, 2017, “Spatial Resolution Enhancement in Hyperspectral Imaging”) discloses a hyperspectral imaging system and method with a pixilated imaging sensor array. U.S. patent application 20180028108 (Shluzas et al., Feb. 1, 2018, “Digital Wound Assessment Device and Method”) discloses a wound assessment device with a camera, additional sensors, and image processing. U.S. patent application 20180039387 (Cheong et al., Feb. 8, 2018, “Method for Controlling Display, Storage Medium, and Electronic Device”) discloses an electronic device including a flexible display.

U.S. patent application 20180074636 (Lee et al., Mar. 15, 2018, “Electronic Device and Method of Controlling Electronic Device”) discloses an electronic device, a method of controlling an electronic device, and a non-transitory computer-readable recording medium. U.S. patent application 20180198982 (Lee et al., Jul. 12, 2018, “Image Capturing Method and Electronic Device”) discloses methods for capturing images using camera-equipped electronic devices and electronic devices. U.S. patent application 20180199856 (Tiwari et al., Jul. 19, 2018, “Electronic Device and Method for Providing Information Related to Skin Type of Object”) discloses an electronic device for providing skin type information by comparison of colors in skin images and non-skin images.

U.S. patent application 20180218637 (Lee et al., Aug. 2, 2018, “Method and Electronic Device for Providing Health Content”) discloses an electronic device with a display, a processor, a communication circuit establishing communication with a server, and a memory storing a specified application. U.S. patent application 20180220952 (Lee et al., Aug. 9, 2018, “Method for Providing Skin Information and Electronic Device for Supporting the Same”) discloses an electronic device to capture a portion of a user's body based on a light source device, a first camera, and a second camera, a memory configured to store a capture image by the image capture device, a display configured to emit light in a specified color at least one region based on driving at least one pixel, and at least one processor configured to be electrically connected with the image capture device, the memory, and the display.

U.S. patent application 20180246591 (Huijser et al., Aug. 30, 2018, “Method of Controlling a Mobile Device”) discloses a method of controlling a mobile device by receiving a time varying current or voltage of a signal generated on the terminals of an acoustic transducer of the mobile device. U.S. patent application 20180249062 (Jin et al., Aug. 30, 2018, “Photographing Method Using External Electronic Device and Electronic Device Supporting the Same”) discloses a photographing method using an external electronic device and an electronic device supporting the same. U.S. patent application 20180279943 (Budman et al., Oct. 4, 2018, “System and Method for the Analysis and Transmission of Data, Images and Video Relating to Mammalian Skin Damage Conditions”) discloses analysis of medical images including one or more objects with a known shape, color characteristic, and size.

U.S. patent application 20180289334 (De Brouwer et al., Oct. 11, 2018, “Image-Based System and Method for Predicting Physiological Parameters”) and U.S. patent Ser. No. 11/026,634 (De Brouwer et al., Jun. 8, 2021, “Image-Based System and Method for Predicting Physiological Parameters”) disclose measuring a physiological parameter by analyzing a facial image. U.S. patent applications 20180293350 (Dimov et al., Oct. 11, 2018, “Image-Based Disease Diagnostics Using a Mobile Device”) and 20190050988 (Dimov et al., Feb. 14, 2019, “Image-Based Disease Diagnostics Using a Mobile Device”), and U.S. patent Ser. No. 10/146,909 (Dimov et al., Dec. 4, 2018, “Image-Based Disease Diagnostics Using a Mobile Device”) disclose a diagnostic system which performs disease diagnostic tests using optical property modifying device and a mobile device. U.S. patent application 20180302564 (Liu et al., Oct. 18, 2018, “System and Apparatus for Co-Registration and Correlation Between Multi-Modal Imagery and Method for Same”) discloses an image capturing device that captures images of a first sensor that includes a first imaging modality, a second sensor that includes a first imaging modality and a third sensor that includes a second imaging modality.

U.S. patent application 20180352150 (Purwar et al., Dec. 6, 2018, “System and Method for Guiding a User to Take a Selfie”) discloses systems and methods for improving the image quality of a selfie using face and landmark detection techniques and face normalization techniques. U.S. patent application 20180374215 (Omer et al., Dec. 27, 2018, “Method and System for Automated Visual Analysis of a Dipstick Using Standard User Equipment”) and U.S. patent Ser. No. 10/559,081 (Omer et al., Feb. 11, 2020, “Method and System for Automated Visual Analysis of a Dipstick Using Standard User Equipment”) disclose a method for analyzing a dipstick using a smartphone. U.S. patent application 20190008694 (Piotrowski et al., Jan. 10, 2019, “Systems and Methods for Wound Healing”) discloses systems and methods to promote wound healing, including a wound dressing having a wound-facing surface and a second surface.

U.S. patent application 20190028634 (Kochler et al., Jan. 24, 2019, “Mobile System and Method”) discloses a mobile system with a camera, wherein the circuitry captures a plurality of images of a user's eye with the camera. U.S. patent application 20190028674 (Smits et al., Jan. 24, 2019, “Holographic Video Capture and Telepresence System”) discloses a system for recording, transmitting, and displaying a three-dimensional image of a face of a user in a video stream. U.S. patent application 20190038135 (Lee et al., Feb. 7, 2019, “Electronic Device, Mobile Terminal and Control Method Thereof”) discloses a mounting portion to which one of a plurality of optical heads is selectively mountable.

U.S. patent application 20190083025 (Aung et al., Mar. 21, 2019, “Devices, Systems, and Methods for Monitoring Wounds”) discloses a wound-monitoring device with an imaging component and a light emitter that emits light at a calibrated wavelength. U.S. patent application 20190244566 (Kim et al., Aug. 8, 2019, “Electronic Device and Method for Compensating Image Quality of Display Based on First Information and Second Information”) discloses a method for compensating for distortion on a display of an electronic device.

U.S. patent application 20190290187 (Adiri et al., Sep. 26, 2019, “Measuring and Monitoring Skin Feature Colors, Form and Size”) and U.S. patent Ser. No. 10/362,984 (Adiri et al., Jul. 30, 2019, “Measuring and Monitoring Skin Feature Colors, Form and Size”) and U.S. Pat. No. 11,026,624 (Adiri et al., Jun. 8, 2021. “Measuring and Monitoring Skin Feature Colors, Form and Size”) disclose kits, diagnostic systems and methods which measure the distribution of colors of skin features by comparison to calibrated colors which are co-imaged with the skin feature. U.S. patent application 20200126227 (Adiri et al., Apr. 23, 2020, “Systems and Methods for Urianlysis Using a Personal Communications Device”) discloses systems and methods for testing visible chemical reactions of a reagent. U.S. patent application 20200211193 (Adiri et al., Jul. 2, 2020, “Tracking Wound Healing Progress Using Remote Image Analysis”) discloses systems and methods for tracking healing progress of multiple adjacent wounds using a colorized surface with colored reference elements.

U.S. patent application 20200211228 (Adiri et al., Jul. 2, 2020, “Uniquely Coded Color Boards for Analyzing Images”) discloses systems and methods for a color board for use in reagent strip testing. U.S. patent Ser. No. 10/991,096 (Adiri et al., Apr. 27, 2021, “Utilizing Personal Communications Devices for Medical Testing”) discloses systems and methods for analyzing visible chemical reactions. U.S. patent application 20210142888 (Adiri et al., May 13, 2021, “Image Processing Systems and Methods for Caring for Skin Features”) discloses systems and methods for image processing of a skin feature which may include receiving first and second images from at least one image sensor associated with a mobile communications device.

U.S. patent application 20190251710 (Park et al., Aug. 15, 2019, “Method and Electronic Device for Converting Color of Image”) discloses methods and apparatuses for displaying a representative color of an image. U.S. patent application 20190307337 (Little et al., Oct. 10, 2019, “Methods and Apparatus for Self-Calibrating Non-Invasive Cuffless Blood Pressure Measurements”) discloses a non-invasive method of measuring blood pressure. U.S. patent applications 20190307400 (Zhao et al., Oct. 10, 2019, “Systems for Personal Portable Wireless Vital Signs Scanner”), 20190350535 (Zhao et al., Nov. 21, 2019, “Systems, Methods, and Apparatus for Personal and Group Vital Signs Curves”), and 20200196962 (Zhao et al., Jun. 25, 2020, “Systems, Methods, and Apparatus for Personal and Group Vital Signs Curves”) disclose methods, systems, and apparatus for periodically and simultaneously scanning for a plurality of vital signs of a user.

U.S. patent application 20190320969 (Levi et al., Oct. 24, 2019, “Bedside or Intra Operative Assessment of Tissue Damage Depth and Readiness for Reconstruction”) discloses a short wave infrared (SWIR) imaging system. U.S. patent application 20190343396 (Khosravi et al., Nov. 14, 2019. “Apparatus for Imaging Skin”) discloses an apparatus with lights and lenses which is attached to a mobile device in order to image skin. U.S. patent application 20190391236 (Downing et al., Dec. 26, 2019, “Photonics Device”) discloses a method of generating separate and discrete wavelengths and light intensity profiles based on an interaction between the separate and discrete wavelengths and a multi-wavelength diffractive optic element.

U.S. patent application 20190391729 (Josephson et al., Dec. 26, 2019, “Apparatuses, Systems, and/or Interfaces for Embedding Selfies into or onto Images Captured by Mobile or Wearable Devices and Method for Implementing Same”) discloses a mobile device with a camera system which captures an image and embeds the image into a background image. U.S. patent application 20200041761 (Sco et al., Feb. 6, 2020, “Optical Lens Assembly and Electronic Device Comprising Same”) discloses an optical lens assembly and electronic apparatus. U.S. patent application 20200059596 (Yoo et al., Feb. 20, 2020, “Electronic Device and Control Method Thereof”) discloses a graphic user interface (GUI) for adjusting the image capture position of a camera. U.S. patent application 20200085164 (Tamir et al., Mar. 19, 2020, “Apparatus with Imaging Functionality”) discloses a camera on a pincer apparatus.

U.S. patent application 20200126283 (Van Vuuren et al., Apr. 23, 2020, “Method and System for Implementing Three-Dimensional Facial Modeling and Visual Speech Synthesis”) disclose tools and techniques for three-dimensional facial modeling and visual speech synthesis. U.S. patent application 20200170564 (Jiang et al., Jun. 4, 2020, “Automatic Image-Based Skin Diagnostics Using Deep Learning”) discloses a deep-learning-based system and method for skin diagnostics. U.S. patent application 20200185073 (De Brouwer et al., Jun. 11, 2020, “System and Method for Providing Personalized Health Data”) discloses a method for providing personalized blood tests.

U.S. patent application 20200186782 (Bigioi et al., Jun. 11, 2020, “Depth Sensing Camera System”) discloses a depth-sensing camera system with a fisheye lens and a (near) infrared sensor. U.S. patent applications 20200193580 (Mccall et al., Jun. 18, 2020, “System and Method for High Precision Multi-Aperture Spectral Imaging”) and 20210082094 (Mccall et al., Mar. 18, 2021, “System and Method for High Precision Multi-Aperture Spectral Imaging”) disclose systems and techniques for spectral imaging using a multi-aperture system with curved multi-bandpass filters positioned over each aperture.

U.S. patent applications 20200193597 (Fan et al., Jun. 18, 2020, “Machine Learning Systems and Methods for Assessment, Healing Prediction, and Treatment of Wounds”) and 20210201479 (Fan et al., Jul. 1, 2021, “Machine Learning Systems and Methods for Assessment, Healing Prediction, and Treatment of Wounds”) disclose machine learning systems and methods for prediction of wound healing. U.S. patent application 20200241290 (Breese et al., Jul. 30, 2020, “Light Deflection Prism for Mounting to a Surface of a Device, Device, and Method for Altering a Field of View of a Camera”) discloses a prism for altering a field of view of a camera.

U.S. patent application 20200279659 (De Brouwer et al., Sep. 3, 2020, “System and Method for Remote Medical Information Exchange”) discloses a method and system for remote medical information exchanging. U.S. patent application 20200293887 (De Brouwer et al., Sep. 17, 2020, “System and Method With Federated Learning Model for Medical Research Applications”) discloses a method and system with a federated learning model for health care applications. U.S. patent application 20200342987 (De Brouwer et al., Oct. 29, 2020, “System and Method for Information Exchange With a Mirror”) discloses a method and system for remote medical information exchange.

U.S. patent application 20200280661 (Barnes et al., Sep. 3, 2020, “Systems and Methods for Spectral Imaging With Compensation Functions”) discloses systems and methods for imaging with compensation functions. U.S. patent application 20200310083 (Kim et al., Oct. 1, 2020, “Optical Lens Assembly and Electronic Device Comprising Same”) discloses an optical lens assembly and electronic device. U.S. patent application 20200330028 (Nejati et al., Oct. 22, 2020, “System and Method for Facilitating Analysis of a Wound in a Target Subject”) discloses a system and method for facilitating analysis of a wound using a neural network. U.S. patent application 20200348493 (Seo et al., Nov. 5, 2020, “Optical Lens System and Electronic Device Including the Same”) discloses an electronic device with a window member, a display panel stacked on a rear surface of the window member, a lens assembly, an iris, and an image sensor.

U.S. patent application 20200352515 (Godavarty et al., Nov. 12, 2020, “Cellphone Based Tissue Oxygenation Measuring Device”) discloses a cellphone-based oxygenation measurement tool. U.S. patent applications 20200352686 (Yancey et al., Nov. 12, 2020, “Scanning Device”) and 20210077233 (Yancey et al., Mar. 18, 2021, “Scanning Device”) disclose a scanning device to scan teeth of a user and acquire images of the teeth. U.S. patent application 20200364862 (Dacosta et al., Nov. 19, 2020, “Wound Imaging and Analysis”) discloses an imaging device using characteristics of wound fluoresce with a unique spectral signature. U.S. patent application 20200374437 (Kang et al., Nov. 26, 2020, “Electronic Device Having Camera Module Capable of Switching Line of Sight and Method for Recording Video”) discloses a camera module capable of changing a sightline and a method of recording video.

U.S. patent application 20200381127 (Silverman et al., Dec. 3, 2020, “Method and System for Assessing, Quantifying, Coding & Communicating Patient's Health and Perioperative Risk”) discloses a multi-dimensional system for assessing, coding, quantifying, displaying, integrating and communicating information relating to patient health and perioperative risk. U.S. patent application 20200401830 (Zhu et al., Dec. 24, 2020, “Method, Apparatus and Storage Medium for Controlling Image Acquisition Component”) discloses a terminal with a movable image acquisition component. U.S. patent application 20200404164 (Wu et al., Dec. 24, 2020, “Method and Apparatus for Controlling Camera, Device and Storage Medium”) discloses a method of controlling a camera applicable to a terminal device with a Dynamic Vision Sensor (DVS) collecting circuit.

U.S. patent application 20210004995 (Burg et al., Jan. 7, 2021, “Methods and Apparatus for Enhancing Color Vision and Quantifying Color Interpretation”) and U.S. patent Ser. No. 11/030,778 (Burg et al., Jun. 8, 2021, “Methods and Apparatus for Enhancing Color Vision and Quantifying Color Interpretation”) disclose a method including selecting a first color sample within a target area in a first image of a first object displayed by a display device; selecting a second color sample within a target area in a second image of a second object displayed in the display device; and comparing the first color sample against the second color sample.

U.S. patent application 20210007606 (Su et al., Jan. 14, 2021, “Method of and Imaging System for Clinical Sign Detection”) discloses a method of and an imaging system for clinical sign detection. U.S. patent application 20210012130 (Park et al., Jan. 14, 2021, “Method and Device for Measuring Biometric Information in Electronic Device”) discloses a method and a device for measuring a user's biometric information in an electronic device and providing information related to the biometric information. U.S. patent application 20210029300 (Lee et al., Jan. 28, 2021, “Electronic Device and Method for Providing Content Associated with Camera Function From Electronic Device”) discloses an electronic device with a display, a camera, a communication module, and a processor.

U.S. patent application 20210084216 (Choi et al., Mar. 18, 2021, “Method of Controlling Camera Device and Electronic Device Thereof”) discloses an electronic device and a method for controlling a camera device in the electronic device based on information input through an adjacent area of the partial area of the display. U.S. patent application 20210124424 (Sandhan et al., Apr. 29, 2021, “Method for Controlling Camera and Electronic Device Therefor”) discloses a method for controlling a camera of an electronic device by generating a plurality of beams using an antenna array.

U.S. patent application 20210127989 (Atamanuk et al., May 6, 2021, “Method of Estimation of the Hemodynamic Status and Capacity of Effort from Patients with Cardiovascular and Pulmonary Diseases”) discloses a method to the effort capacity of a human individual as an expression of distance walked by the individual. U.S. patent application 20210128021 (Emalfarb et al., May 6, 2021, “Systems and Methods for Verified Biomeasurements”) discloses a method for generating, via a camera, image data that is reproducible as an image of at least a portion of a subject.

U.S. patent application 20210132795 (Kurbanova et al., May 6, 2021, “Smart Mirror and Table Top Devices with Sensor Fusion of Camera Vision, Acoustic, and Multi-Point Capacitive Touch Control”) discloses a smart mirror. U.S. patent application 20210136263 (Pitman, May 6, 2021, “Mobile Terminal”) discloses a mobile terminal with a camera and at least one distal lens disposed remotely from the camera. U.S. patent application 20210145359 (Hunt et al., May 20, 2021, “Wound Analysis Device and Method”) discloses a monitoring and therapy system which collects video images of a tissue site, amplifies said video images via Eulerian Video Magnification, and determines a treatment parameter.

U.S. patent applications 20210161621 (Salah et al., Jun. 3, 2021, “Method for Analysing a Dental Situation”) and 20210186658 (Salah et al., Jun. 24, 2021, “Method for Analysing a Dental Situation”) disclose a method for analyzing a patient's dental situation. U.S. patent application 20210217797 (Hashiguchi et al., Jul. 15, 2021, “Solid-State Imaging Device and Electronic Apparatus”) discloses an imaging device with a first substrate having a pixel unit. U.S. patent application 20210218923 (Yoda et al., Jul. 15, 2021, “Solid-State Imaging Device and Electronic Device”) discloses an imaging device including an array unit in which a plurality of pixels each have a photoelectric conversion unit. U.S. patent application 20210225032 (Hain et al., Jul. 22, 2021, “Multiple Camera Calibration”) discloses a method for generating pose transformation data between first and second rigidly mounted digital cameras having non-coincident fields of view.

U.S. patent application 20210225509 (Wang et al., Jul. 22, 2021, “Method, Apparatus and System for Guiding Diagnosis-And-Treatment, and Computer Readable Storage Medium”) discloses a method, apparatus and system for guiding diagnosis-and-treatment, and a computer readable storage medium. U.S. patent application 20210225516 (Liu et al., Jul. 22, 2021, “Method and Device for Processing Physiological Data, and Storage Medium”) discloses a method for processing physiological data, a device for processing physiological data, and a non-volatile storage medium. U.S. patent application 20210226906 (Ryu et al., Jul. 22, 2021, “Electronic Device and Method for Image Control Thereof”) discloses a device with a display, a communication interface, and a processor comprising processing circuitry.

U.S. patent application 20210227019 (Soon-Shiong et al., Jul. 22, 2021, “Camera-to-Camera Interactions, Systems and Methods”) discloses methods of delegating media capturing functionality from one device to another. U.S. patent application 20210227413 (Yang et al., Jul. 22, 2021, “Method and Communication Device for Performing Measurement”) discloses a method for performing measurement using a configured measurement gap (MG) from a serving cell.

The following section reviews relevant art from sources other than patents and patent applications.

Ashique, 2015, “Clinical Photography in Dermatology Using Smartphones: An Overview,” Indian Dermatology Online Journal, 2015, 6, 3, 158, provides an overview of smartphone photography in clinical dermatology. Burns, 2015, “Digital Photography and the Medical Selfie,” Journal of Participatory Medicine, Feb. 11, 2015, 7, e3, discusses how capturing and controlling health data can promote a patient from being a passive recipient to an active co-creator of modern health care. Burns, 2019, “Creating Consumer-Generated Health Data: Interviews and a Pilot Trial Exploring How and Why Patients Engage,” Journal of Medical Internet Research, 2019, 21(6), e12367, discusses how consumer-generated health data (CGHD) is being used by consumers and how it influences their engagement via a validated framework. Coleman, 2019, “Cell Phone Based Colorimetric Analysis for Point-of-Care Settings.” The Analyst, Jan. 28, 2019, 144(6), 1935-1947, discusses a cell phone imaging algorithm that enables analysis of assays that would typically be evaluated via spectroscopy.

Diethei, 2018, “Using Smartphones to Take Eye Images for Disease Diagnosis in Developing Countries,” Proceedings of the Second African Conference for Human Computer Interaction: Thriving Communities (Windhoek, Namibia) (AfriCHI '18). ACM, New York, NY, Article 34, discusses EyeGuide, a mobile application that helps people to take eye images. Diethei, 2020, “Medical Selfies: Emotional Impacts and Practical Challenges,” MobileHCI '20: 22nd International Conference on Human-Computer Interaction with Mobile Devices and Services, Oct. 5, 2020, 8, 1-2, discusses how medical images taken with mobile phones by patients can allow screening, monitoring, and diagnosis of skin lesions. Farr, 2020, “Healthy.io, Maker of a Medical Selfie, Is Part of the New Generation of Israeli Health-Tech Companies,” CNBC, Jun. 22, 2020, discusses innovations by Healthy.io including an FDA-approved stick which is dipped into urine and calibration stickers which allow clinicians to accurately measure wounds.

Florida Atlantic University, 2019, “Selfies to Self-Diagnosis: Algorithm Amps Up Smartphones to Diagnose Disease.” Science Daily, Feb. 12, 2019, discusses image capture using three smartphones: the Android Moto G; the iPhone 6; and the Samsung Galaxy Edge. Granot, 2008, “A New Concept for Medical Imaging Centered on Cellular Phone Technology,” PloS one. 3, e2075, discusses the use of cell phones to transmit remote medical imaging data. Healthy.io, 2020, “Digitizing Wound Management: A Standardized, Evidence-Based Approach to Healing,” Healthy.io, White Paper, 2020, discusses Healthy.io's wound management technology which enables clinicians to take automatic measurements using a smartphone. Lee, 2018, “Recent Trends in Teledermatology and Teledermoscopy.” Dermatology Practical & Conceptual, 2018, 8, 3, 214, discusses recent trends in teledermatology via both asynchronous store-and-forward modes and a real-time video conferencing modes.

Mai, 2020, “The Effect of Perioral Scan and Artificial Skin Markers on the Accuracy of Virtual Dentofacial Integration: Stereophotogrammetry Versus Smartphone Three-Dimensional Face-Scanning.” International Journal of Environmental Research and Public Health, 2020, 18(1), 229, discusses a study of the effects of different matching methods on the accuracy of dentofacial integration in stereophotogrammetry and smartphone face-scanning systems. Otero, 2019, “Comparison of Different Smartphone Cameras to Evaluate Conjunctival Hyperaemia in Normal Subjects,” Scientific Reports, 2019, 9, 1339, compares the objective and subjective quantification of conjunctival redness in images obtained with calibrated and non-calibrated cameras, in different lighting conditions and optical magnifications. Peng. 2017, “Constructing a 3D Multiple Mobile Medical Imaging System through Service Science, Management, Engineering and Design,” Systems, 2017, 5(1), 5, discusses a 3D multiple mobile medical imaging system (3D MMMIS) for doctor's diagnosis and treatment.

Pires, 2015, “Wound Area Assessment Using Mobile Application,” Biodevices, 2015, discusses the use of mobile devices to identify wound contours and areas in captured images. Pivo, 2019. “Pivo Pod Get Insanely Creative Gifs, Photos, Videos,” Indiegogo, campaign closed Jan. 19, 2019, provides an overview of the “Pivo” pod, a phone-holding device with a stationary base and pivoting upper portion, which pivots a phone to track a moving (e.g. dancing) person in order to create videos (e.g. for sharing on social media). Senthilkumaran, 2021. “Simple Technique for Medical Photography in the Emergency Department During the COVID Pandemic: Say Cheese,” Journal of Emergency Medicine, Letter to the Editor, 5/1/2021, 60(5), e135, discusses mounting a smartphone on a selfie stick for use in an emergency room.

Wicklund, 2019, “mHealth Researchers Look to Make the Selfie a Health Resource.” mHealth Intelligence, Aug. 9, 2019, discusses the use of medical selfies to evaluation blood pressure, atrial fibrillation, and other medical conditions. Wong, 2018, “Real-World Use of Telemedicine: A Picture Is Worth a Thousand Words,” PMFA Journal, 5(2), December/January, 2018, discusses information technology issues related to the use of mobile phone images within the NHS system. Zhou, 2012, “A Survey of Optical Imaging Techniques for Assessing Wound Healing,” International Journal of Intelligent Control and Systems, September, 2012 17(3), 79-85, discusses how optical imaging can be used to overcome both the lack of objectivity and noninvasiveness in the identification, evaluation, and assessment of cutaneous wounds and cutaneous wound healing.

### SUMMARY OF THE INVENTION

This invention can be embodied in a method of using a cellphone to capture images of a portion of a patient's body. Machine learning and/or artificial intelligence can be used provide real-time guidance to a person concerning how to move a cellphone along an arcuate path over a portion of a patient's body to capture images of the portion from different angles and distances. This invention can also be embodied in a cellphone-moving device with a base, a pivoting arm which is attached to the base by an axle, and a cellphone dock which is part of the pivoting arm. A cellphone is removably-attached to the cellphone dock. The cellphone is then moved by the arm in an arcuate path in space over a portion of a patient's body in order to capture images of the portion from different angles and distances. The images resulting from this method and/or device can be integrated together to create a digital 3D image of the portion of the person's body. In an example, machine learning and/or artificial intelligence can be used to analyze the digital 3D image. In an example, a remote healthcare provider can examine the digital 3D image from different angles and distances.

### BRIEF INTRODUCTION TO THE FIGURES

FIG. 1. shows an example of how a cellphone can be moved in an arcuate path over a portion of a patient's body to capture images of the portion of the body from different angles and distances.

FIGS. 2 and 3 show two different side views of a cellphone-moving device with a pivoting arm to which a cellphone is attached.

FIGS. 4 and 5 show two close-up side views of the cellphone-moving device shown in FIGS. 2 and 3.

FIG. 6 shows how the length of a pivoting arm can be adjusted.

FIG. 7 shows how adjustment of the length of a pivoting arm can move a cellphone along a serpentine oscillating path.

FIGS. 8 and 9 show two different side views of a cellphone-moving device with a centrally-connected moving arm to which a cellphone is attached.

FIGS. 10 and 11 show two different side views of cellphone-moving device with a revolving wheel to which a cellphone is attached.

FIGS. 12 and 13 show two different side views of cellphone-moving device with two arches which span a portion of a person's body.

FIG. 14 shows a cellphone dock and how a cellphone can be removably-attached to the dock.

FIG. 15 shows movement of a cellphone along a serpentine oscillating path with an overall perimeter which is shaped like a paper fan.

### DETAILED DESCRIPTION OF THE FIGURES

In an example, a method of using a cellphone to capture images of a portion of a patient's body can comprise: using machine learning and/or artificial intelligence to guide a person concerning how to move a cellphone along an arcuate path over a portion of a patient's body to capture images of the portion from different angles and distances; and using machine learning and/or artificial intelligence to integrate the images together to create a digital 3D image of the portion.

In an example, the person can be guided concerning how to move the cellphone along the arcuate path by machine-generated auditory cues. In an example, the auditory cues can be spoken directions. In an example, the person can be guided concerning how to move the cellphone along the arcuate path by machine-generated visual cues. In an example, the visual cues can be displayed via augmented reality. In an example, the method can further comprise using machine learning and/or artificial intelligence to analyze the digital 3D image. In an example, the method can further comprise enabling a remote healthcare provider to examine the digital 3D image from different angles and distances.

In an example, a method of using a cellphone to capture images of a portion of a patient's body can comprise: using a cellphone-moving device to move a cellphone along an arcuate path over a portion of a patient's body to capture images of the portion from different angles and distances; and using machine learning and/or artificial intelligence to integrate the images together to create a digital 3D image of the portion. In an example, the arcuate path can be a section of a circle. In an example, the cellphone-moving device can include a pivoting arm to which the cellphone is attached. In an example, the pivoting arm can pivot back and forth like a windshield wiper.

In an example, a cellphone-moving device can comprise: a base; a pivoting arm which is attached to the base by an axle; and a cellphone dock which is part of the pivoting arm, wherein a cellphone is removably-attached to the cellphone dock, and wherein the cellphone is moved by the arm in an arcuate path in space over a portion of a patient's body in order to capture images of the portion from different angles and distances.

In an example, the pivoting arm can be attached to the base at a location on the lower half of the arm. In an example, the cellphone dock can be attached to the arm at a location on the upper half of the arm. In an example, the cellphone dock can extend out from the arm in a perpendicular manner. In an example, the device can further comprise one or more light emitters. In an example, the length of the pivoting arm can be adjustable. In an example, the device can further comprise an electrical motor which moves the pivoting arm. In an example, the device can further comprise a data processor, a data receiver, and a data transmitter. In an example, the device can be in electromagnetic communication with the cellphone.

In an example, a method of using a cellphone to capture images of a portion of a patient's body to be analyzed by artificial intelligence and/or examined by a remote healthcare provider can comprise: moving a cellphone along an arcuate path in space over (e.g. over, above, across, and/or around) a portion of a patient's body to capture images of the portion from different angles and distances, wherein a camera of the cellphone has a first focal vector when the cellphone is in a first location on the arcuate path and the first focal vector is directed toward the portion of the patient's body, and wherein the camera of the cellphone has a second focal vector when the cellphone is in a second location on the arcuate path and the second focal vector is directed toward the portion of the patient's body; and integrating (e.g. integrating, combining, compiling, and/or processing) the images together to create a digital 3D image of the portion which is then analyzed by artificial intelligence and/or examined by a remote healthcare provider. In an example, the cellphone can be moved manually by a person with real-time (auditory, visual, or tactile) guidance. In an example, the cellphone can be moved automatically by a specialized cellphone-moving device. In an example, the cellphone can be moved automatically by a general-purpose home robot.

In an example, a method of using a cellphone to capture images of a portion of a patient's body to be analyzed by artificial intelligence and/or examined by a remote healthcare provider can comprise: guiding a person in real time concerning how to move a cellphone along an arcuate path in space over (e.g. over, above, across, and/or around) a portion of a patient's body to capture images of the portion from different angles and distances, wherein a camera of the cellphone has a first focal vector when the cellphone is in a first location on the arcuate path and the first focal vector is directed toward the portion of the patient's body, and wherein the camera of the cellphone has a second focal vector when the cellphone is in a second location on the arcuate path and the second focal vector is directed toward the portion of the patient's body; and integrating (e.g. integrating, combining, compiling, and/or processing) the images together to create a digital 3D image of the portion which is then analyzed by artificial intelligence and/or examined by a remote healthcare provider. In an example, the person can be guided in real time by machine-generated auditory, visual, or tactile cues.

In an example, a method of using a cellphone to capture images of a portion of a patient's body to be analyzed by artificial intelligence and/or examined by a remote healthcare provider can comprise: using a cellphone-moving device to move a cellphone along an arcuate path in space over (e.g. over, above, across, and/or around) a portion of a patient's body to capture images of the portion from different angles and distances, wherein a camera of the cellphone has a first focal vector when the cellphone is in a first location on the arcuate path and the first focal vector is directed toward the portion of the patient's body, and wherein the camera of the cellphone has a second focal vector when the cellphone is in a second location on the arcuate path and the second focal vector is directed toward the portion of the patient's body; and integrating (e.g. integrating, combining, compiling, and/or processing) the images together to create a digital 3D image of the portion which is then analyzed by artificial intelligence and/or examined by a remote healthcare provider. In an example, the cellphone-moving device can be real time electronic communication with a remote server.

In an example, images captured by a cellphone of a portion of a patient's body from different angles and distances can be integrated (e.g. integrated, combined, compiled, and/or processed) together by machine learning and/or artificial intelligence in order to create a digital 3D image (e.g. virtual 3D model) of the portion. In an example, machine learning methods and/or artificial intelligence can be used to analyze the digital 3D image to evaluate the possibility of a skin abnormality and/or assess a wound. In an example, a digital 3D image can be examined to evaluate the possibility of a skin abnormality or assess a wound by a remote (e.g. non-local) healthcare provider. In an example, a digital 3D image can be first analyzed by machine learning and/or artificial intelligence and then, if indicated based on the results of the automated analysis, examined by a healthcare provider.

In an example, a method of remote (e.g. remote, virtual, and/or online) evaluation of a portion of a patient's body for medical (e.g. diagnostic or therapeutic) purposes can comprise: receiving images of a portion of a patient's body from different angles and distances which have been captured by a cellphone which was moved over (e.g. over, across, or around) the portion in an arcuate path in space; using machine learning and/or artificial intelligence to integrate (e.g. integrate, combine, compile, or process) these images together into a digital 3D image of the portion of the patient's body; and making the digital 3D image accessible for a healthcare provider to examine virtually from different angles and distances. In an example, the digital 3D image aways remains in focus as the healthcare provider examines it from different angles and distance.

In an example, a cellphone can be moved manually by a person who receives real-time machine-generated auditory, visual, and/or tactile guidance. In an example, this real-time guidance can comprise auditory (e.g. spoken), visual (e.g. augmented reality display), or tactile (e.g. vibration) cues (e.g. cues or directions) which are provided to the person in real time by means of machine learning methods and/or artificial intelligence. In an example, this guidance can be adjusted (e.g. adjusted, modified, adapted) in real time based on analysis of the images being captured by the cellphone using machine learning and/or artificial intelligence. In another example, a cellphone can be moved by a specialized cellphone-moving device. In an example, the cellphone can be removably-attached to a moving arm or wheel of a cellphone-moving device. In an example, movement of the cellphone by this device can be adjusted (e.g. adjusted, modified, adapted) in real time by machine learning methods and/or artificial intelligence based on analysis of images being captured by the moving cellphone.

In an example, a digital 3D image of a portion of a patient's body which has been created by integrating images which were captured by a cellphone can be used for remote dermatologic examination (e.g. teledermatology). In an example, this examination can help to identify skin cancer. In an example, a digital 3D image can also be used for remote examination of a wound. In an example, this examination can help to identify whether a wound is healing or getting worse over time. In an example, a chronological sequence of digital 3D images of the same portion of a patient's body can be assembled and used to evaluate changes in the portion over time for better assessment of changes in a skin abnormality and/or wound healing progress.

In this disclosure, visual remote examination of a patient is defined as analysis of images of that patient by a non-local machine (e.g. machine learning and/or artificial intelligence in a remote server) and/or examination of those images by a non-local healthcare provider, wherein a non-local location can be many miles away from the location of the patient. In an example, remote examination of a digital 3D image of a portion of a patient's body can occur long after the raw images have been captured. In an example, remote examination can include analysis of (changes in) the location, size, shape, color, and texture of an abnormality or wound on a portion of a patient's body. In an example, examination can include using machine learning and/or artificial intelligence to analyze (changes in) the location, size, shape, color, texture, growth, and/or healing of an abnormality or wound before examination and review by a healthcare provider. In an example, remote examination can include analysis of changes in the location, size, shape, color, and texture of an abnormality or wound on a portion of a patient's body by analyzing a chronological sequence of digital 3D images of the same portion.

In an example, raw images of a portion of a patient's body captured by a cellphone from different angles and distances can be digitally integrated (e.g. integrated, combined, or compiled) to create a digital 3D image of the portion. In an example, this integration can be done by machine learning methods and/or artificial intelligence. In an example, remote examination of a digital 3D image of a portion of a patient's body can occur asynchronously, some time after the raw images have been captured. In an example, remote examination of the portion can occur days, weeks, or months after the raw images have been captured. In an example, a digital 3D image of the portion can allow a healthcare provider to zoom in on a particular area of the portion and/or pan around the area to view it from different angles, with the area still remaining in focus.

In an example, a digital 3D image of a portion of a patient's body created by moving a cellphone in an arcuate path in space over that portion can be analyzed in a series of hierarchical steps. A first step can be analysis of the digital 3D image by machine learning methods and/or artificial intelligence. A second step can be examination of the digital 3D image by a human healthcare provider. In an example, a second step of human evaluation may be done only if indicated by the results of the first step of automated evaluation. In an example, human examination may be triggered only if automated analysis (by machine learning methods or artificial intelligence) indicates a high probability of an abnormality. In an example, a first step of an examination can be analysis via machine learning methods and/or artificial intelligence and a second step of examination can be review by a human healthcare provider, wherein the second step is pursued only if the first step indicates a probability of an abnormality above a selected probability level.

In an example, a cellphone can be moved manually by a person in order to capture images of a portion of a patient's body. In an example, this person can receive real-time machine-generated auditory, visual, or tactile guidance concerning how to move the cellphone. Alternatively, a cellphone can be moved by a specialized cellphone-moving device which has been specifically designed for this purpose. In an example, a specialized cellphone-moving device can be designed to move a cellphone in an arcuate path in space over (e.g. over, across, or around) a portion of a patient's body. In an example, a specialized cellphone-moving device can include a pivoting and/or rotating arm to which a cellphone is removably-attached, wherein movement of the arm moves the cellphone. This arm can move back and forth like the arm of a metronome or a windshield wiper. In an example, a specialized cellphone-moving device can include a revolving and/or rotating wheel to which a cellphone is removably-attached, wherein rotation of the wheel moves the cellphone.

In an example, a specialized cellphone-moving device can be sufficiently portable and light-weight that it can be sent to a patient's home to provide remote imaging capability in support of remote care (e.g. virtual care, online visits, and/or telemedicine). In an example, a health plan or healthcare provider can ship a specialized cellphone-moving device to a patient's home in advance of a specific remote care visit (e.g. virtual visit, online visit, or telemedicine visit) or at the start of a coverage period. In another example, a health plan or healthcare provider can give a specialized cellphone-moving device to a patient during an in-person care visit for subsequent use in the patient's home.

In an example, a cellphone can be moved in an arcuate path in space over a portion of a patient's body by generic hardware, such as general-purpose home robot. In an example, a home robot can have a movable arm which can grasp a cellphone or to which a cellphone can be removably-attached. In an example, a general-purpose home robot can be operated using specialized software to move cellphone in an arcuate path of the portion of the patient's body. In an example, a general-purpose home robot can be guided (e.g. programmed) to move a cellphone in an arcuate path over a portion of a patient's body in accordance with the method embodiments disclosed herein. In an example, a general-purpose home robot can be programmed to grasp, move, and operate a cellphone in accordance with the method embodiments disclosed herein.

Methods, devices, or systems such as those disclosed herein which enable capturing medical-quality 3D images of a portion of a patient's body at home can make it easier (e.g. less expensive and less time consuming) to create a chronological sequence of images of that portion for longitudinal analysis of changes. In an example, scanning the same portion of a patient's body can be conveniently done from home on a periodic basis (e.g. every week, month, or year). A chronological sequence of images of the same portion over time can be useful for evaluating changes in a skin abnormality or a wound.

In an example, digital 3D images of the same portion of a patient's body which are created periodically over a long period of time (e.g. several weeks, months, or years) can be compared and/or combined into a time series to identify changes in the portion. In an example, changes in the portion over time can be analyzed to evaluate potential growth, which can indicate skin cancer. In an example, changes in a wound over time can be analyzed to evaluate healing or worsening of the wound. In an example, machine learning methods and/or artificial intelligence can be used to analyze such changes in a portion of a patient's body over time.

In an example, machine learning methods and/or artificial intelligence can be used to align digital 3D images of a portion of a patient's body which have been captured at different times over a long period (e.g. over several months or even years). In an example, digital 3D images of a portion of a patient's body based on images captured at different times can be integrated to create a chronological digital 4D image (with time being the fourth dimension) wherein a healthcare provider examining the model can view changes in the portion over time as well as view the portion from different angles and distances. For example, a provider can navigate to a particular area of a digital 4D image and then activate a chronological shift (e.g. by sliding a virtual bar) which changes the time of the image. Further, comparison of changes over time can be automated. For example, a digital 4D image can display changes in the outline (e.g. perimeter) of an abnormality as a series of (labeled or color-coded) contour lines, like the chronological version of altitude contour lines on a geographic map.

In an example, machine learning methods and/or artificial intelligence can be used to guide a person in real time (e.g. with auditory, visual, or tactile cues and/or directions) concerning how to move and/or position a cellphone relative to a portion of a patient's body in order to align a current image of this portion with a past image of this portion. In an example, augmented reality displayed on a cellphone or via smart eyewear can provide visual cues which guide a person concerning how to move and/or position a cellphone relative to a portion of a patient's body in order to align a current image of the portion with a past image of the portion.

In an example, a cellphone can be moved in an arcuate path in space over a portion of a patient's body in order to capture raw images of a wound, injury, skin lesion, and/or tissue abnormality on that portion from different angles and distances. In an example, these raw images can be integrated (e.g. integrated, combined, compiled, and/or processed) together by machine learning methods and/or artificial intelligence to create a digital 3D image of the wound, injury, skin lesion, and/or tissue abnormality which can be virtually examined by a remote healthcare provider. In an example, this digital 3D image of a wound, injury, skin lesion, and/or tissue abnormality can be analyzed using machine learning and/or artificial intelligence before examination by a healthcare provider in order to highlight unusual features for consideration by the provider.

In an example, a cellphone can be moved along an arcuate path in space above a portion of a patient's body to capture images of that portion from different angles and distances. In an example, this arcuate path can be shaped like a section of a circle (e.g. a semicircle). In an example, the center of this circle can be on the surface of the portion of the patient's body which is being examined. In an example, the center of the circle can be within the interior of the portion of the patient's body being examined. In an example, an arcuate path in space along which a cellphone is moved can be between one-quarter and one-half of a circle, wherein the center of the circle is on the surface of the portion of the patient's body being examined. In an example, an arcuate path can have a spiral shape. In an example, an arcuate path can have a helical shape.

In an example, an arcuate path in space along which a cellphone is moved can have a concave shape, wherein a surface of the portion of the patient's body being examined is within the concavity. In an example, an arcuate path in space along which a cellphone is moved can have a parabolic shape, wherein the focal point of this parabola is on the surface of the portion of the patient's body being examined. In an example, this arcuate path can have an undulating shape. In an example, this arcuate path can have a sinusoidal undulating shape. In an example, this arcuate path can have a shape which is the combination of a sinusoidal shape and an arch shape, wherein this shape has an overall fan-shaped perimeter and a serpentine interior.

In an example, a cellphone can be moved along an arcuate path in space over a portion of a patient's body to capture images of the portion from different angles. In an example, this arcuate path can have a concave shape. In an example, this arcuate path can be a section (e.g. an arc) of a circle. In an example, this arcuate path can have a semicircular shape. In an example, this arcuate path can have a shape which is between 25% and 50% of a circle. In an example, this arcuate path can have a conic section shape. In another example, this arcuate path can have a parabolic shape. In an example, this arcuate path can have a helical and/or spiral shape.

In an example, a person moving a cellphone over a portion of a patient's body can be guided concerning how to move the cellphone along a spiral path in space, wherein the center of the spiral is directly over the (center of the) portion of the patient's body. In an example, the center of the spiral can be directly over an abnormality on the portion of the patient's body. In an example, a person moving a cellphone over a portion of a patient's body can be guided to move the cellphone along a helical path in space, wherein the center of the helix is directly over the (center of the) portion of the patient's body. In an example, the center of the helix can be directly over an abnormality on the patient's body.

In an example, a specialized cellphone-moving device can move, tilt, pivot, rotate, and/or wave a cellphone over a portion of a patient's body. In an example, a cellphone-moving device can move, tilt, pivot, rotate, and/or wave a cellphone in an arc (e.g. section of a circle or ellipse) over a portion of a patient's body. In an example, a cellphone-moving device can move, tilt, pivot, rotate, and/or wave a cellphone in a circle over a portion of a patient's body. In an example, a cellphone-moving device can move, tilt, pivot, rotate, and/or wave a cellphone in a spiral over a portion of a patient's body.

In an example, an oscillating arcuate path in space along which a cellphone is moved can curve closer to a portion of the patient's body with each side-to-side (e.g. back and forth) movement, thereby creating a serpentine (e.g. undulating and fan-shaped) path in space. In an example, this undulating and fan-shaped path can be in a virtual plane which is orthogonal to the plane of the patient's body. In an example, such an arcuate path can have a fan-shaped or keystone-shaped perimeter (e.g. like the area of a car windshield that is cleared by one windshield wiper) and an undulating (e.g. serpentine) interior.

In an example, a cellphone can be moved along an arcuate path in space over a portion of a patient's body to capture images of the portion from different angles. In an example, the cellphone can be a constant distance from the portion of the patient's body as it moves along the arcuate path. In an example, the arcuate path itself can be a constant distance from the portion of the patient's body. In another example, the distance between a cellphone and a portion of the patient's body can vary as the cellphone moves along an arcuate path. In an example, the distance between the arcuate path itself and the portion of the patient's body can vary along the length of the path. In an example, the distance between an arcuate path and a portion of the patient's body can be between 2 inches and 3 feet. In an example, the distance between an arcuate path and a portion of the patient's body can be between 4 inches and 2 feet.

In an example, there can be constant distance between points on an arcuate path in space (along which a cellphone is moved) and the closest surface of a portion of a patient's body to be examined. In an example, there can be constant distance between points on an arcuate path in space (along which a cellphone is moved) and the cross-sectional center of a portion of a patient's body to be examined. In another example, the distances between points on an arcuate path in space (along which a cellphone is moved) and the closest surface of a portion of a patient's body to be examined can decrease as the cellphone moves along the arcuate path in a first direction and then further decrease as the cellphone moves along the arcuate path in a second (e.g. opposite) direction.

In an example, a patient can lie on a horizonal surface (e.g. bed, couch, or floor) in their home as a portion of their body (e.g. head, arm, or leg) is imaged by a moving cellphone. One can define a virtual vertical plane which is orthogonal to this horizontal surface and which also passes through this portion of the patient's body. In an example, a cellphone can move along an arcuate path in space over this portion of the body, wherein the cellphone is at a first location along the path at a first time, wherein the cellphone is at second location along the path at a second time, and wherein the vertical plane is between the first location and the second location. In an example, the first and second locations can be the same distance from the portion of the body.

In an example, a cellphone can be oscillated back and forth (e.g. first moved in one direction and then moved in the opposite direction) along an arcuate path in space over a portion of a patient's body. In an example, the arcuate path can comprise a single line, wherein the cellphone double's back on itself with each oscillation. In this manner, the cellphone captures images of the portion from different angles, but the same distance. In another example, the arcuate path can move closer to the patient's body with each oscillation. In this manner, the cellphone captures images of the portion from different angles and progressively-smaller distances.

In an example, one side of a cellphone can be selected from which to capture images of a portion of a patient's body. In an example, this side can be tilted (e.g. tilted or rotated) as the cellphone moves along an arcuate path so that this side remains substantially-parallel to a virtual plane which is tangential and/or parallel to the nearest surface of the portion of the patient's body. In an example, when the cellphone is moved manually by a person, then this tilting is guided by machine-generated real-time (auditory, visual, or tactile) directions given to the person. In an example, when the cellphone is moved by a cellphone-moving device, then this tilting can be done automatically by the device.

In an example, when a cellphone is moved in an arcuate path in space to capture images of a portion of a patient's body, then the side of the cellphone with the active camera can be kept pointed toward the portion of the patient's body. In an example, the cellphone can be tilted (e.g. tilted or rotated) as it is moved along the arcuate path so that the focal vector of the active camera is always pointed toward the portion of the patient's body.

In an example, a cellphone camera can have a focal vector which is substantially orthogonal to the side of the cellphone on which the camera is located. In an example, a cellphone camera can have a first focal vector (in a first direction) when the camera is at a first location along the arcuate path and a second focal vector (in a second direction) when the camera is at a second location along the arcuate path. In an example, the second location can be at least 6 inches from the first location. In an example, the first and second focal vectors can intersect at a point on a portion of a patient's body. In an example, the second location can be at least 2 feet from the first location. In an example, the first and second focal vectors can intersect on the surface of a portion of a patient's body. In an example, the first and second focal vectors (or linear extensions of them) can intersect within the portion of a patient's body.

In an example, a cellphone can be moved along an arcuate path in space over a portion of a patient's body in order to capture images of that portion from different angles and distances. In an example, this can be done by having the cellphone record continuous video while it moves along the arcuate path. In another example, this can be done by having the cellphone capture a plurality of still images, from different angles and distances, as it moves along the arcuate path. In an example, a cellphone can be set to capture still images at periodic time intervals as it moves along the arcuate path. In an example, a cellphone can be set to capture still images at selected locations as it moves along the arcuate path. In an example, control of the cellphone's camera can be temporarily transferred to a remote system during the imaging process so that the system can trigger images at selected times or locations.

In an example, a cellphone can a capture a plurality of still images of a portion of a patient's body at selected times as the cellphone moves along an arcuate path above the portion. In an example, a cellphone can a capture a plurality of still images of a portion of a patient's body at selected periodic time intervals (e.g. a selected number of images per second or per minute) as the cellphone moves along an arcuate path above the portion. In an example, a cellphone can a capture a plurality of still images of a portion of a patient's body at selected locations along the path as the cellphone moves along an arcuate path above the portion. In an example, a cellphone can capture a plurality of still images of a portion of a patient's body at selected distance intervals (e.g. every inch or every couple inches) along the path as the cellphone moves along an arcuate path above the portion. In an example, a cellphone can capture a plurality of still images of a portion of a patient's body at selected angles relative to the portion. In an example, a cellphone can capture a plurality of still images of a portion of a patient's body at selected distances relative to the portion.

In an example, a cellphone can capture continuous video of a portion of a patient's body as the cellphone moves along an arcuate path above the portion. In an example, this video can be segmented, extracted, or parsed into a series of still images and these still images can then be integrated into a digital 3D image of the portion. In an example, this video can be directly integrated into a digital 3D image of the portion by machine learning methods and/or artificial intelligence. In an example, this video can be directly integrated into a digital 3D image of the portion, wherein this digital 3D image is a still image, but can be viewed interactively, from different angles and distances, by a healthcare provider. In an example, multiple images of the portion from the same angle can be digitally combined by machine learning and/or artificial intelligence to increase clarity of the view of the portion from that angle.

In an example, a method of supporting remote examination of a portion of a patient's body for medical (e.g. diagnostic or therapeutic) purchases can comprise: guiding a person concerning how to move a cellphone relative to a selected portion of a patient's body in order to capture a plurality of clear, in-focus images of that portion from different angles and distances; receiving the plurality of images; using machine learning and/or artificial intelligence to integrate (e.g. integrate, combine, compile, or assemble) those the plurality of images together into a digital 3D image of the portion of the patient's body; and making the digital 3D image accessible for a healthcare provider to examine from different angles and distances.

In an example, a method for creating a digital 3D image of a portion of a patient's body to be examined by a remote healthcare provider can comprise: guiding a person how to move a cellphone along an arcuate path in space over a portion of a patient's body; and integrating a plurality of images of the portion of the patient's body captured by the cellphone from different angles and distances into a digital 3D image of the portion of the patient's body which is then examined by a remote healthcare provider.

In an example, a method for creating a digital 3D image of a portion of a patient's body to be examined by a remote healthcare provider can comprise: using machine-generated (auditory, visual, or tactile) cues to guide a person concerning how to move a cellphone along an arcuate path in space over a portion of a patient's body; and integrating a plurality of images of the portion of the patient's body captured by the cellphone from different angles and distances into a digital 3D image of the portion of the patient's body which is then examined by a remote healthcare provider.

In an example, a method for creating a digital 3D image of a portion of a patient's body to be examined by a remote healthcare provider can comprise: using machine-generated vocal directions to guide a person concerning how to move a cellphone along an arcuate path in space over a portion of a patient's body; and integrating a plurality of images of the portion of the patient's body captured by the cellphone from different angles and distances into a digital 3D image of the portion of the patient's body which is examined by a remote healthcare provider.

In an example, a cellphone can be moved manually along an arcuate path in space over a portion of a patient's body by the patient (or by a person who is with the patient). In an example, the cellphone can be moved manually along an arcuate path in space over a portion of a patient's body by the patient (or by a person who is with the patient) wherein the person moving the cellphone receives machine-based (auditory, visual, or tactile) guidance concerning how to keep the cellphone on the path.

In an example, a cellphone can be moved along an arcuate path in space over a portion of a patient's body. In an example, the cellphone can be moved manually by a person (e.g. either the patient or another person who is with the patient). In an example, the person moving the phone can be guided by machine-generated (auditory, visual, or tactile) cues or directions concerning how to move the cellphone along the arcuate path. In an example, auditory cues or directions can comprise machine-generated (e.g. AI synthesized) vocal directions. In an example, machine learning methods and/or artificial intelligence can be used: to track the location and/or movement of the cellphone relative to the portion of the patient's body; and to generate vocal cues or directions to guide the person moving the cellphone to keep it on the right path. In an example, these vocal cues or directions can be given to the person through the cellphone's speaker.

In an example, machine-generated real-time guidance to help a person move a cellphone along an arcuate path in space over a portion of a patient's body can be adapted, adjusted, and/or modified based on real-time machine-based analysis of the images of the portion which are being captured by the cellphone. In an example, machine-based analysis of these images can include analysis of their focus and/or angle. In an example, if an image is out-of-focus because the cellphone is too far away from the portion, then real-time guidance can instruct the person to move the cellphone closer. On the other hand, if an image is out-of-focus because the cellphone is too close to the portion, then real-time guidance can instruct the person to move the cellphone farther out. In an example, if images do not provide sufficient angular perspective, then real-time guidance can instruct the person to move the cellphone in a wider arcuate path.

In an example, a cellphone can be moved manually along an arcuate path by a person, wherein the person's movements are guided in real-time by vocal directions from a remote healthcare provider (e.g. via the phone) concerning how to move the cellphone to keep it along the arcuate path. In an example, a cellphone can be moved manually along an arcuate path by a person, wherein the person's movements are guided in real-time by machine-generated vocal directions (e.g. via the phone) concerning how to move the cellphone to keep it along the arcuate path.

In an example, a cellphone can be moved manually by a person in an arcuate path in space over a portion of a patient's body to capture images of that portion from different angles and distances. Those images can then be compiled into a digital 3D image of that portion of the patient's body for virtual examination by a healthcare provider. In an example, a method of moving the cellphone can include guiding (e.g. in real time) the person moving the phone concerning how to move the cellphone via machine-generated vocal directions. These vocal directions tell the person how to move the cellphone.

In an example, a cellphone can be moved manually by a person along an arcuate path over a portion of a patient's body, wherein the person is guided by real-time machine-generated vocal (e.g. spoken) directions concerning how to move the cellphone. For example, machine-generated spoken directions can include expressions such as: “move the phone slowly in a semi-circle around the arm,” “move the phone to your right”, or “move the phone around an inch closer to the arm.”

In an example, a cellphone can be moved manually along an arcuate path in space by a person, wherein this person is guided by machine-generated real-time vocal directions (e.g. spoken directions, suggestions, or commands) concerning how to move the cellphone along the arcuate path. For example, machine-generated vocal directions can include things like—“move the phone to the other side of the person's arm”. “keep the camera facing toward the arm”, “gradually move the phone closer to the person's arm as you move it to the right.” “not bad performance for a human.” “don't forget to floss your teeth,” or “good job keeping the phone a constant distance from the arm.”

In an example, a cellphone can be moved manually by a person along an arcuate path in space, wherein the person moving the phone receives real-time guidance from the cellphone concerning how to move the cellphone. In an example, the real-time guidance can be machine generated (e.g. generated by machine learning methods and/or artificial intelligence). In an example, the real-time guidance can be auditory, such as machine-generated directions spoken from the cellphone speaker. In an example, the real-time guidance can be visual, such as machine-generated cues shown on the cellphone screen. In an example, a cellphone can be moved manually by a patient who receives real-time guidance via machine-generated vocal directions such as—“move the cellphone closer to the patient”, “move the cellphone to your right”, or “tilt the phone more toward you”.

In an example, a method for guiding a person to move a cellphone in an arcuate path over a portion of a patient's body can include using machine learning and/or artificial intelligence (AI) to track the location of the cellphone relative to the portion; and generate vocal directions for the person to keep the cellphone moving along the arcuate path in space. For example, these machine-generated vocal directions could include expressions such as—“move the phone to your right”, “move the phone a little closer to the arm”. “you can't touch this”, or “keep the phone camera pointed toward the arm.”

In an example, a cellphone can be moved manually by a person in an arcuate path in space over a portion of a patient's body to capture images of that portion from different angles and distances. These images can then be digitally integrated into digital 3D image of that portion for virtual examination by a remote healthcare provider. In an example, a method of guiding the person concerning how to move the cellphone can be done using sound cues. In an example, the frequency, pitch, and/or volume of sound emitted from the cellphone can change if the cellphone deviates from the arcuate path, thereby helping the person to keep the cellphone along the arcuate path. This method is similar, in some respects, to the way in which the rate of beeps emitted from a Geiger counter changes with movement toward or away from a source of radiation.

In an example, a cellphone can be moved manually by a person, wherein the person is guided though sound cues concerning how to move the cellphone along an arcuate path in space over a portion of a patient's body. These sound cues can include changes in sound frequency, pitch, and/or volume. These changes in sound frequency, pitch, and/or volume can inform the person concerning how (e.g. in which direction) to move the cellphone to keep it on the arcuate path in space.

In an example, a cellphone can be moved manually along an arcuate path in space by a person with guidance from machine-generated sound patterns. In an example, the person can be directed to move the cellphone in a selected direction (e.g. right, left, up, down) based on a selected sound pattern (e.g. higher frequency, lower frequency, louder sound, softer sound). In another example, the person can be directed to keep the cellphone moving along the arcuate path in space by sound patterns triggered by deviation from this path. The latter is similar to a car lane tracking system which triggers a warning sound if the car deviates from its lane.

In an example, a method of moving a cellphone along an arcuate path in space over a portion of a patient's body can include using visual cues to guide a person (e.g. the patient or another person with the patient) concerning how to move the cellphone. In an example, a digital display on the cellphone or smart eyewear can display an augmented reality (AR) image which guides the person. In an example, the augmented reality image can show a digital representation of the cellphone and the arcuate path superimposed on the actual an image of the portion. In an example, the augmented reality image can show a virtual representation of the arcuate path superimposed over an image of the portion and the cellphone. In an example, the person can use this augmented reality to guide their movement of the cellphone along the path over the portion. In an example, the color of the virtual arcuate path shown can change, depending on whether the cellphone is closer or father from the path. This color changing feature can help guide the person to keep the cellphone on the path.

In an example, a method of capturing images of a portion of a patient's body for examination by a remote healthcare professional can include moving a cellphone manually in an arcuate path through space over the portion of the patient's body. In an example, this method can include guiding a person moving the cellphone (e.g. the patient or another person with the patient) though visual cues (e.g. augmented reality) concerning how to move the cellphone along the arcuate path, wherein a virtual image of how the cellphone should be moved and/or the arcuate path in space is shown via augmented reality. In an example, these augmented reality cues can be displayed on a cellphone. In an example, these augmented reality cues can be shown in the person's field of view by smart eyewear (e.g. augmented reality eyewear).

In an example, a method for moving a cellphone in an arcuate path in space over a portion of a patient's body can include guiding a person (e.g. the patient or another person with the patient) concerning how to move the cellphone. In an example, a person can be guided though visual cues concerning how to move a first cellphone along the arcuate path, where a virtual image of how the first cellphone should be moved (e.g. showing a virtual image of its arcuate path superimposed over the portion) is shown via augmented reality on the display of a second cellphone.

In an example, a method for creating a digital 3D image of a portion of a patient's body to be examined by a remote healthcare provider can comprise: using an augmented reality display to guide a person concerning how to move a cellphone along an arcuate path in space over a portion of a patient's body; and integrating a plurality of images of the portion of the patient's body captured by the cellphone from different angles and distances into a digital 3D image of the portion of the patient's body which is examined by a remote healthcare provider. In an example, the augmented reality display can be shown on the screen of a cellphone. In an example, the augmented reality image can be shown in a person's field of view of augmented reality eyewear.

In an example, a cellphone can be moved manually along an arcuate path over a portion of a patient's body, wherein the patent (or other person moving it) is guided by visual cues in real-time concerning how to move the cellphone to keep it along the arcuate path. In an example, these visual cues can be displayed via augmented reality. For example, an augmented reality display on a cellphone or on augmented reality eyewear can show the arcuate path being superimposed over the portion of the patient's body. The person uses this augmented reality display to guide their movement of the cellphone along the arcuate path.

In an example, a cellphone can be moved manually by a person, wherein this person receives guiding though visual cues concerning how to move the cellphone along the arcuate path. In an example, these visual cues can be superimposed over the portion of the patient's body in an augmented reality image shown on the cellphone screen or via smart eyewear. In an example, a cellphone can be moved by a person manually in an arcuate path in space over a portion of a patient's body. In an example, the person can be guided though visual cues concerning how to move the cellphone along the arcuate path, wherein the virtual cues are displayed on the cellphone screen. In another example, these virtual cues can be displayed via augmented reality eyewear. In an example, augmented reality eyewear can show the arcuate path in space virtually in the person's field of view so that the person can move the cellphone along this path.

In an example, a cellphone can be moved manually by a person along an arcuate path in space over a portion of a patient's body in order to capture images of the portion from different angles and distances. In an example, the person moving the cellphone can receive auditory, visual, and/or vibrational machine-based guidance in real time concerning how to keep to move the cellphone to keep it on the right path. In an example, this visual guidance can include displaying the arcuate path as a virtual object in an augmented reality display, wherein this display also shows the portion of the patient's body. In an example, this visual guidance can show the person moving the cellphone how to move it relative to the portion of the patient's body.

In an example, a first cellphone can be moved manually along an arcuate path in space over a portion of a patient's body by a person, wherein the person is guided by an augmented reality display shown on a second cellphone which shows the arcuate path. In an example, the screen of the second cellphone can show the location of the first cellphone along the arcuate path in a manner similar to the “flight tracker” displays available on airplane flights which show where the plane is along a flight path on a virtual map. In an example, this augmented reality display can show the portion of the patient's body in the background, similar to the virtual map in the background of a “flight tracker” display.

In an example, a cellphone can be moved by a specialized cellphone-moving device instead of a person. In an example, a cellphone-moving device can be sufficiently light-weight, portable, and inexpensive that it can be easily shipped to a patient's home for capturing medical images at home. In an example, a specialized cellphone-moving device can operate in a relatively self-contained manner during a scan and then transmit images to a remote server for integration into a digital 3D image. In another example, a specialized cellphone-moving device can be in real-time electronic communication with a remote data processor during a scan. Real-time communication can able a feedback loop wherein movement of the cellphone over a portion of a patient's body is adjusted in real-time based on analysis of images being transmitted by the cellphone.

In an example, a method of supporting remote evaluation of a portion of a patient's body for medical (e.g. diagnostic or therapeutic) purchases can comprise: sending a specialized cellphone-moving device to a patient's home; instructing a person at the home concerning how to attach a cellphone to the device and how to position the device relative to a portion of a patient's body; receiving a plurality of images captured by the cellphone as it is moved over the portion of the patient's body by the cellphone-moving device; using machine learning and/or artificial intelligence to integrate (e.g. integrate, combine, compile, or assemble) the plurality of images into a digital 3D image; and making the digital 3D image accessible for a healthcare provider to examine from different angles and distances.

In an example, a method for creating a digital 3D image of a portion of a patient's body for evaluation by a remote healthcare provider can include: sending a specialized cellphone-moving device to a patient's home; instructing the patient or other person concerning how to attach a cellphone to the device and position the device relative to a selected portion of the patient's body; receiving images of the portion of the patient's body from the cellphone; analyzing these images to determine how the cellphone should be moved in order to capture clear images of the portion of the patient's body from different angles and distances; remotely controlling the device to move the cellphone in order to capture clear images of the portion of the patient's body from different angles and distances; and integrating (e.g. integrating, combining, compiling, or assembling) these images to create a digital 3D image of the portion of the patient's body for evaluation by a remote healthcare provider.

In an example, a method for creating a digital 3D image of a portion of a patient's body for evaluation by a remote healthcare provider can include: sending a cellphone-moving device to a patient's home; instructing a person (the patient or someone else) to attach a cellphone to the cellphone-moving device; receiving images of a selected portion of the patient's body from the cellphone; using machine learning and/or artificial intelligence to analyze these images in real time; remotely controlling movement of the cellphone-moving device in order to move the cellphone so as to capture clear images of the portion of the patient's body from different angles and distances; and using machine-learning and/or artificial intelligence to integrate (e.g. integrate, combine, compile, or assemble) these images to create a digital 3D image of the portion of the patient's body for diagnostic and/or therapeutic purposes.

In an example, a method for creating a digital 3D image of a portion of a patient's body to be examined by a remote healthcare provider can comprise: sending a device to a patient's home, wherein the device is capable of moving a cellphone in an arcuate path in space over a portion of the patient's body; and integrating a plurality of images of the portion of the patient's body captured by the cellphone from different angles and distances into a digital 3D image of the portion of the patient's body which is examined by a remote healthcare provider.

In an example, a cellphone-moving device can enable a person to take a better medical selfie by enabling better control of image angle, distance, focus, lighting, and/or color calibration.

In an example, a person can set up such a device by attaching a conventional camera-equipped mobile phone to the device and connecting the device to the internet. Alternatively, such a device can be used at a remote medical facility, enabling a non-local healthcare specialist to view and evaluate a portion of a patient's body. In an example, a phone-moving device can serve as the connectivity component of a remote medical imaging system, wherein a conventional mobile phone serves as the imaging component. In an example, a health plan or provider can ship a cellphone-moving device to a person's home in conjunction with a virtual visit. In another example, a health plan or provider can give such a device to a patient during an in-person visit for the patient to take and use at home throughout the year.

In an example, a specialized cellphone-moving device can have a moving (e.g. pivoting) arm. In an example, a cellphone can be removably-attached to the upper half (e.g. upper end) of the arm. In an example, this arm can pivot back and forth like a windshield wiper or the arm of a metronome. In an example, an actuator pivots the arm back and forth like a windshield wiper or the arm of a metronome. In this manner, the arm moves the cellphone in an arcuate path in space. As the arm pivots, it moves the attached cellphone along an arcuate path in space. When the device is placed in proximity to a selected portion of a patient's body so that the cellphone is over that portion, movement of the arm causes the cellphone to move in an arcuate path in space over that portion of the patient's body. In this manner, the cellphone captures images of the portion of the patient's body from different angles and distances. These images are then used to create a digital 3D image of that portion of the patient's body for remote evaluation.

In an example, a cellphone can be moved by a device along an arcuate path in space over a portion of a patient's body. In an example, the cellphone can be removably-attached to the device, which then moves the cellphone along the acuate path. In an example, the device can have a pivoting arm which waves back and forth (e.g. oscillates) like the arm of a metronome (or windshield wiper), except that the arm is sufficiently large to hold a cellphone. In an example, the cellphone can be attached to the upper half of the arm. In an example, the lower half arm can be attached to the rest of device (e.g. by an axel and/or joint). In an example, a motor in the device can pivot the arm back and forth, thereby causing the cellphone to move back and forth along an arcuate path. In an example, the speed of movement of the arm (and also the cellphone) can be controlled by the device. In an example, the length of the arm can be changed and controlled by the device.

In an example, a cellphone can be moved along an arcuate path in space, wherein the centroid of the cellphone stays on the path and a side of the cellphone stays tangential to the path. In an example, a cellphone can be moved back and forth (e.g. first in one direction and then in the opposite direction) along an arcuate path in space which is over a portion of a patient's body. In an example, a cellphone can be moved in a first direction and then in a second (e.g. opposite) direction along an arcuate path in space which is over a portion of a patient's body.

In an example, a cellphone can be removably-attached to a moving (e.g. pivoting) arm of a cellphone-moving device, wherein this arm moves the cellphone along an arcuate path in space. In an example, the size of the arcuate path can be adjusted by adjusting the length of the arm. In an example, the length of the arm can be adjusted manually. In an example, the length of the arm can be adjusted automatically by the device. In an example, the length of the arm can be adjusted once, before a series of arm oscillations (e.g. movements back and forth). In an example, the length of the arm can be adjusted multiple times during a series of arm oscillations. In an example, the length of the arm can be adjusted at least once during each oscillation of the arm.

In an example, a cellphone-moving device can comprise a telescoping arm to which a cellphone is attached. In an example, a cellphone-moving device can comprise a moving (pivoting and telescoping) arm to which a cellphone is attached. In an example, a cellphone-moving device can comprise a cantilevered telescoping arm to which a cellphone is attached. In an example, the portable phone-moving device can further comprise a cantilevered arm which holds the phone out over the portion of the patient's body. In an example, a cellphone-moving device can comprise a movable flexibly-segmented gooseneck arm to which a cellphone is attached. In an example, a cellphone-moving device can further comprise wheels or treads which enable the base of the device to move along a flat surface.

In an example, a cellphone-moving device can comprise: a base; an actuator (e.g. electrical motor) in the base; a data processing unit; a wireless data transmitter and receiver; a pivoting arm which is movably-attached to the base, wherein the actuator pivots the arm back and forth (like a windshield wiper or an arm on a metronome); and a cellphone dock on the pivoting arm, wherein a cellphone can be removably-attached to the cellphone dock, and wherein movement of the pivoting arm causes an attached cellphone to move in an arcuate path in space.

In an example, a device which moves a cellphone in an arcuate path in space can comprise a pivoting arm. In an example, the device can further comprise a phone-docking component which is part of (or attached to) the upper half of the pivoting arm. In an example, the virtual plane which best fits the phone-docking component can be perpendicular to the longitudinal axis of the pivoting arm. In an example, the phone-docking component can hold the cellphone perpendicular to the longitudinal axis of the pivoting arm. In an example, the phone-docking component can hold the cellphone out from the arm so that the cellphone can extend out from the arm over a portion of a patient's body.

In an example, a device for moving a cellphone in an arcuate path over a portion of a patient's body can comprise a phone-docking component, a pivoting arm, and a motor. In an example, a cellphone can be removably attached to the phone-docking component. In an example, the best-fitting virtual plane of the cellphone can be substantially parallel to the best-fitting virtual plane of the phone-docking component when the cellphone is attached to the phone-docking component. In an example, the best-fitting virtual plane of the phone-docking mechanism can be perpendicular to the longitudinal axis of the pivoting arm. In an example, the pivoting arm can be pivoted (e.g. waved back and forth) by the motor.

In an example, a device for moving a cellphone in an arcuate path in space can include a moving arm to which the cellphone is removably-attached. In an example, device can move (e.g. pivot or wave) the arm (back and forth) like a metronome arm or a windshield wiper. In an example, the cellphone can trace out a section of a circle in space as it is moved when the arm moves. In an example, the cellphone can extend out from the arm so that it extends out into space over a portion of a patient's body. In an example, the device can further comprise a phone-docking mechanism which extends out from the arm by a distance between 3 inches and 1 foot.

In an example, a cellphone-moving device can comprise: a weighted base; an actuator (e.g. electrical motor) in the base; a data processing unit; a wireless data transmitter and receiver; a pivoting arm extends upward from the base and is movably-attached to the base, wherein the actuator pivots the arm (e.g. moving it back and forth like a windshield wiper or the arm on a metronome); and a cellphone dock which is part of (or attached to) the upper half of the pivoting arm, wherein cellphone dock is substantially perpendicular to the longitudinal axis of pivoting arm, wherein a cellphone can be removably-attached to the cellphone dock, and wherein movement of the pivoting arm causes an attached cellphone to move in an arcuate path in space.

In an example, a cellphone-moving device can comprise: a base; an actuator (e.g. electrical motor) in the base; a data processing unit; a wireless data transmitter and receiver; a pivoting arm which is movably-attached to the base, wherein the actuator pivots the arm back and forth (like a windshield wiper or arm on a metronome); and a cellphone dock which is on the pivoting arm, wherein cellphone dock is perpendicular to the pivoting arm, wherein the cellphone dock extends out at least 3 inches from the pivoting arm, wherein a cellphone can be removably-attached to the cellphone dock, and wherein movement of the pivoting arm causes an attached cellphone to move in an arcuate path in space.

In an example, a device which moves a cellphone in an arcuate path in space can include a pivoting arm. In an example, this pivoting arm can pivot back and forth like the arm of a metronome or a windshield wiper. In an example, the lower half of the pivoting arm can be moveably attached (e.g. via an axel) to a base. In an example, the upper half of the pivoting arm can include a cellphone dock to which a cellphone can be removably attached. In an example, a cellphone can be removably-attached to the cellphone dock with a mechanism selected from the group consisting of: clip, clasp, clamp, snap, pin, hook, strap, and magnet. In an example, the pivoting arm can be a straight rod. In an example, the pivoting arm can be a curved rod. In an example, the pivoting arm can have flexibly-attached sections (e.g. be a gooseneck arm).

In an example, a cellphone can be removably-attached to a cellphone-moving device which moves a cellphone along an arcuate path in space. In an example, the cellphone can be removably attached to the device by one or more clips. In an example, the cellphone can be removably attached to the device by one or more clasps or clamps. In an example, the cellphone can be removably attached to the device by one or more hooks. In an example, the cellphone can be removably attached to the device by one or more pins or plugs. In an example, the cellphone can be removably attached to the device by one or more straps.

In an example, a cellphone can be attached to a cellphone dock on a cellphone-moving device by a mechanism selected from the group consisting of: clip, clasp, clamp, prong, plug, snap, hook, band, strap, hook-and-eye material. In an example, a cellphone can be attached to a cellphone dock by one or more hooks or prongs. In an example, a cellphone can be attached to a cellphone dock by one or more straps, bands, or ties. In an example, a cellphone can be attached to a cellphone dock by hook-and-eye material (e.g. Velcro™). In an example, a cellphone can be attached to a cellphone dock by one or more magnets. In an example, a cellphone dock can include a pocket, recess, track, or other opening into which a mobile phone is inserted.

In an example, a cellphone can be attached to a cellphone-moving device by an attachment mechanism selected from the group consisting of: one or more clips, clasps, clamps, hooks, straps, bands, recesses, pockets, magnets, and hook-and-eye materials. In an example, a cellphone can be attached to a cellphone dock on a cellphone-moving device by an attachment mechanism selected from the group consisting of: one or more clips, clasps, clamps, hooks, straps, bands, recesses, pockets, magnets, and hook-and-eye materials.

In an example, a cellphone-moving device can include a cellphone dock to which a cellphone is attached. In an example, this cellphone dock is designed so that it does not obscure the cellphone's distal-facing camera. In an example, this cellphone dock is designed so that it does not block the cellphone's proximal-facing screen. In an example, a cellphone dock can latch, clamp, or otherwise hold, a cellphone against a flat surface on the cellphone dock. In an example, a cellphone dock can be a rectangular recess, opening, pocket or track into which a cellphone is inserted. In an example, there can be a wire electronic connection (e.g. plug and/or wire) between the cellphone dock and the cellphone. In an example, there can be a wire electronic connection (e.g. plug and/or wire) between the cellphone dock and the cellphone.

In an example, a cellphone dock (to which a cellphone is removably-attached) on a cellphone-moving device can further comprise one or more light emitters (e.g. LEDs). In an example, these light emitters can be activated to increase the illumination of the portion of a patient's body which is being imaged by the cellphone. In an example, the intensity of light emitted from these light emitters can be increased to increase the illumination of the portion of a patient's body which is being imaged by the cellphone. In an example, the spectrum of light emitted by these light emitters can be adjusted to change the spectrum of light illuminating the portion of the patient's body. In an example, these light emitters can be activated to adjust the angle of illumination of the portion of a patient's body which is being imaged by the cellphone.

In an example, a cellphone dock (to which a cellphone is removably-attached) on a cellphone-moving device can further comprise one or more light emitters (e.g. LEDs) and one or more light receivers, wherein the light emitters and receivers together comprise one or more spectroscopic sensors. In an example, the light emitters and receivers together can enable spectroscopic analysis of a portion of a patient's body from which light is reflected. In an example, light from the light emitters can be reflected by a portion of a patient's body and received by the light receivers, wherein changes in the spectral distribution of this light caused by this reflection are used to analyze the molecular composition of the portion of the patient's body. In an example, light from the light emitters can be reflected by a portion of a patient's body and received by the light receivers, wherein changes in the spectral distribution of this light caused by this reflection are analyzed as part of medical evaluation of the portion of the patient's body.

In an example, a device for moving a cellphone in an arcuate path over a portion of a patient's body can: receive images from the cellphone in real time; analyze the images to identify how the cellphone should be moved to capture clear images of the portion of the patient's body from different angles and distances; and move the cellphone based on this analysis. In an example, a cellphone-moving device for moving a cellphone in an arcuate path over a portion of a patient's body can comprise: an image-analysis component which uses machine learning and/or artificial intelligence to analyze images of a portion of a patient's body captured by a cellphone over the portion; and an actuator which moves the cellphone in response to the results of this analysis to ensure that the cellphone captures a plurality of clear images of the portion from different angles and distances.

In an example, a system for moving a cellphone in an arcuate path in space to capture images of a portion of a patient's body from different angles and distances can include a real-time feedback loop, wherein images from the cellphone are analyzed in real time to guide movement of the cellphone. Such a real-time feedback loop can help to ensure that images are clear (e.g. in focus), capture the intended area, and include views from multiple angles. In an example, images can be analyzed in real-time by machine learning methods and/or artificial intelligence.

In an example, a method for creating a digital 3D image of a portion of a patient's body can include: instructing a person to attach a cellphone to cellphone-moving device and guiding the person concerning how to position the cellphone in proximity to a selected portion of a patient's body; receiving images of the portion of the patient's body from the cellphone as the cellphone moves relative to the portion; using machine learning and/or artificial intelligence to analyze these images to determine how movement of the cellphone should be adjusted in order to capture clear images of the portion of the patient's body from different angles and distances; controlling the cellphone-moving device to move the cellphone in order to capture clear images of the portion of the patient's body from different angles and distances; and using machine-learning and/or artificial intelligence to integrate (e.g. integrate, combine, compile, or assemble) these images together to create a digital 3D image of the portion of the patient's body for evaluation by machine learning methods and/or a remote healthcare provider.

In an example, a method for creating a digital 3D image of a portion of a patient's body can include: instructing a patient to attach a cellphone to cellphone-moving device and to position the cellphone in proximity to a selected portion of the patient's body; receiving images of the portion of the patient's body from the cellphone; using machine learning and/or artificial intelligence to analyze these images to determine how the cellphone should be moved relative to the portion of the patient's body in order to capture clear, in-focus images of the portion of the patient's body from different angles and distances; remotely controlling the cellphone-moving device to move the cellphone in order to capture clear, in-focus images of the portion of the patient's body from different angles and distances; and using machine-learning and/or artificial intelligence to integrate (e.g. integrate, combine, compile, or assemble) these images to create a digital 3D image of the portion of the patient's body for diagnostic and/or therapeutic purposes.

In an example, a cellphone-moving device which moves a cellphone along an arcuate path in space can further comprise one or more light emitters. In an example, one or more of these light emitters can be to the right of the cellphone and one or more of these light emitters can be to the left of the cellphone. In an example, one or more of these light emitters can be on a pivoting arm to which the cellphone is removably-attached. In an example, one or more of these light emitters can be on the device base to which the pivoting arm is movably attached. In an example, one or more of these light emitters can be on a moving arm which is different from the moving arm to which a cellphone attached, thereby enabling illumination of images captured by the cellphone from different angles.

In an example, different light emitters on a cellphone-moving device can emit light toward a portion of a patient's body at different wavelengths, spectral frequencies, and/or colors. In an example, a subset of light emitters on a cellphone-moving device can be selected for activation based on analysis of which wavelength, spectrum, and/or color of illumination would be most helpful for examination of the portion by a healthcare provider. For example, a first set of images of the portion of a patient's body can be captured primarily with light in the visual range of the spectrum and a second set of images may be captured primarily with light in the near-infrared range of the spectrum. In an example, these images can be used to create digital 3D images which have been illuminated by light with different spectral distributions.

In an example, one or more light emitters on a cellphone-moving device can emit light along projection vectors which are not parallel with the focal vector of a camera on a cellphone which is attached to the device. In an example, one or more light emitters on a cellphone-moving device can emit light along projection vectors which intersect the focal vector of a cellphone camera at an angle between 10 degrees and 40 degrees. In an example, one or more light emitters on a cellphone-moving device can emit light along projection vectors which intersect the focal vector of a cellphone camera at an angle between 30 degrees and 70 degrees.

In an example, a cellphone-moving device can include a distance finder. In an example, a cellphone-moving device can include an infrared light emitter and an infrared light receiver, wherein the emitter and receiver together comprise a mechanism to measure the distance between the cellphone (dock) and the portion of a patient's body which is imaged. In an example, a cellphone dock on a cellphone-moving device can project a light beam which helps align the focal vector of the cellphone's camera with a portion of a patient's body which is imaged. In an example, this projected light beam can be a low-power laser beam like that of laser pointer. In an example, the light beam can turn off automatically after the cellphone dock has been properly positioned over the portion of the patient's body so that it does not interfere with images of the portion.

In an example, a device which moves a cellphone along an arcuate path in space can further comprise one or more light emitters. In an example, then intensity of light from the light emitters can be adjusted in real time (e.g. within seconds) based on machine analysis of images captured by the cellphone. In an example, the selection of which light emitters are activated at a given time can be adjusted in real time based on machine analysis of images captured by the cellphone. In an example, the spectral distribution (e.g. wavelength, frequency, or color) of light from the light emitters can be adjusted in real time based on machine analysis of images captured by the cellphone.

In an example, a cellphone-moving device can further comprise one or more light-emitters (such as LEDs). The device can be part of a system which uses machine learning and/or artificial intelligence to analyze images captured by the cellphone in real time. If these images are too dim, then the one or more light emitters may be automatically activated or their light intensity can be automatically increased. If these images are too light, then the one or more light emitters may be automatically decreased.

In an example, a cellphone-moving device can have an electromagnetic motor which moves (e.g. rotates, revolves, pivots, rolls, slides, swings, oscillates, extends, and/or drives) a cellphone which is attached to the device, thereby changing the focal angle, direction, location, and/or distance of the cellphone's camera. In an example, a cellphone-moving device can move a cellphone along the surface of a virtual sphere, wherein a portion of a patient's body which is being imaged is located at the center of the virtual sphere. In an example, a cellphone-moving device can sequentially move a cellphone sequentially along the surfaces of a plurality of concentric virtual spheres, wherein a portion of a patient's body being imaged is located at the center of each of the virtual spheres.

In an example, a cellphone-moving device can further comprise a light projector which projects a visual fiducial marker in proximity to the portion of a patient's body which is being imaged. In an example, a light projector can project a point or pattern of laser light. In an example, a projected fiducial marker can be used in image analysis to determine the distance and/or size of an abnormality on the portion of the patient's body. For example, a projected laser beam with known dimensions (at a given distance) which appears in an image of an abnormality can be used to calibrate the size of the abnormality. Also, a projected laser beam with a known two-dimensional pattern (e.g. circle, square, or dot array) which appears in an image of an abnormality can be used to identify the view angle of the image. Also, a projected laser beam with a known color spectrum which appears in an image of an abnormality can be used to correct any color errors caused by a camera filter (or other type of automated image processing). In an example, a light projector which projects a visual fiducial marker can be located on a phone-docking component of a device. In an example, a light projector which projects a visual fiducial marker can be located on a moving arm of the device.

In an example, a person can be guided how to move a cellphone in 3D space relative to a portion of a patient's body by augmented reality. In an example, an augmented reality display on a cellphone or eyewear can display a virtual pointer or cursor which directs the person where to move the cellphone along an arcuate path in space. In an example, an augmented reality display can show the portion of the patient's body to be imaged with a virtual pointer, cursor, or line path superimposed in 3D space above the portion.

In an example, a cellphone can be moved by a home robot to capture images of a portion of a patient's body. In an example, a cellphone can be attached the arm of a home robot or a home robot arm can grasp the cellphone. Then the robot moves the cellphone in an arcuate path over the portion of the patient's body. In an example, a home robot can grasp a cellphone and move it along an arcuate path in space over a portion of a patient's body. In an example, the robot can track the location of the cellphone relative to the portion of the patient's body in order to maintain a constant distance or vary the distance in a selected manner as the cellphone is moved back and forth. In an example, movement of a home robot can be remote controlled by machine learning methods and/or artificial intelligence to capture images of a portion of a patient's body from different angles and distances for medical evaluation purposes.

In an example, a method of capturing images of a portion of a patient's body with a cellphone can include software which, with owner permission, enables a remote healthcare provider to temporarily adjust the settings (e.g. aperture, image filters, automatic color adjustments, ISO, or shutter speed) of the cellphone camera and/or activate the flash to improve the usefulness of captured images for medical purposes. In an example, a method of capturing images of a portion of a patient's body with a cellphone can include software which, with owner permission, enables an imaging system to temporarily adjust the settings (e.g. aperture, image filters, automatic color adjustments, ISO, or shutter speed) of the cellphone camera and/or activate the flash to improve the usefulness of captured images for medical purposes.

In an example, a cellphone can capture a plurality of still images of a portion of a patient's body as the cellphone is moved in an arcuate path in space over the portion. In another example, a cellphone can capture continuous video images of the portion of a patient's body as the cellphone is moved in an arcuate path in space over the portion. Whether the capture images are a plurality of still images or image frames from a continuous video, these images can be integrated (e.g. integrated, combined, compiled, or processed) into a digital 3D images of the portion of the patient's body which can be examined later by a remote healthcare provider.

Currently, cellphones have much better image capture and display capabilities than smart watches (or other wrist-worn devices), so the device and method embodiments discussed herein focus on moving cellphones along an arcuate path in space over a portion of a patient's body to capture images of the portion from different angles and distances. However, if smart watches (or other wrist-worn devices) evolve in the future to have excellent image capture capabilities, then it is to be understood that a smart watch can be substituted for a cellphone in the device and method embodiments disclosed herein.

In an example, Artificial Intelligence (AI) can be used to provide guidance to a person concerning how to move a cellphone over a portion of a patient's body. In an example, Artificial Intelligence (AI) can be used to integrate images of a portion of a patient's body captured from different angles and distances into a digital 3D image of the portion. In an example, least squares estimation can be used to provide guidance to a person concerning how to move a cellphone over a portion of a patient's body. In an example, logistic discrimination can be used to provide guidance to a person concerning how to move a cellphone over a portion of a patient's body. In an example, machine learning can be used to provide guidance to a person concerning how to move a cellphone over a portion of a patient's body.

In an example, multivariate analysis can be used to provide guidance to a person concerning how to move a cellphone over a portion of a patient's body. In an example, multivariate linear regression can be used to provide guidance to a person concerning how to move a cellphone over a portion of a patient's body. In an example, Fourier transformation can be used to provide guidance to a person concerning how to move a cellphone over a portion of a patient's body. In an example, pattern recognition can be used to provide guidance to a person concerning how to move a cellphone over a portion of a patient's body.

In an example, principle components analysis can be used to provide guidance to a person concerning how to move a cellphone over a portion of a patient's body. In an example, random forest analysis can be used to provide guidance to a person concerning how to move a cellphone over a portion of a patient's body. In an example, fuzzy logic can be used to provide guidance to a person concerning how to move a cellphone over a portion of a patient's body. In an example, inductive logic programming can be used to provide guidance to a person concerning how to move a cellphone over a portion of a patient's body. In an example, a support vector machine can be used to provide guidance to a person concerning how to move a cellphone over a portion of a patient's body.

In an example, association rule learning can be used to provide guidance to a person concerning how to move a cellphone over a portion of a patient's body. In an example, linear discriminant analysis can be used to provide guidance to a person concerning how to move a cellphone over a portion of a patient's body. In an example, Bayesian analysis can be used to provide guidance to a person concerning how to move a cellphone over a portion of a patient's body. In an example, deep learning algorithms can be used to provide guidance to a person concerning how to move a cellphone over a portion of a patient's body. In an example, an artificial neural network can be used to provide guidance to a person concerning how to move a cellphone over a portion of a patient's body.

In an example, data analytics can be used to provide guidance to a person concerning how to move a cellphone over a portion of a patient's body. In an example, time-series analysis can be used to provide guidance to a person concerning how to move a cellphone over a portion of a patient's body. In an example, logistic discrimination can be used to integrate images of a portion of a patient's body captured from different angles and distances into a digital 3D image of the portion. In an example, multivariate analysis can be used to integrate images of a portion of a patient's body captured from different angles and distances into a digital 3D image of the portion.

In an example, Bayesian analysis can be used to integrate images of a portion of a patient's body captured from different angles and distances into a digital 3D image of the portion. In an example, multivariate linear regression can be used to integrate images of a portion of a patient's body captured from different angles and distances into a digital 3D image of the portion. In an example, association rule learning can be used to integrate images of a portion of a patient's body captured from different angles and distances into a digital 3D image of the portion. In an example, pattern recognition can be used to integrate images of a portion of a patient's body captured from different angles and distances into a digital 3D image of the portion.

In an example, principle components analysis can be used to integrate images of a portion of a patient's body captured from different angles and distances into a digital 3D image of the portion. In an example, inductive logic programming can be used to integrate images of a portion of a patient's body captured from different angles and distances into a digital 3D image of the portion. In an example, random forest analysis can be used to integrate images of a portion of a patient's body captured from different angles and distances into a digital 3D image of the portion.

In an example, time-series analysis can be used to integrate images of a portion of a patient's body captured from different angles and distances into a digital 3D image of the portion. In an example, least squares estimation can be used to integrate images of a portion of a patient's body captured from different angles and distances into a digital 3D image of the portion. In an example, linear discriminant analysis can be used to integrate images of a portion of a patient's body captured from different angles and distances into a digital 3D image of the portion.

In an example, a support vector machine can be used to integrate images of a portion of a patient's body captured from different angles and distances into a digital 3D image of the portion. In an example, data analytics can be used to integrate images of a portion of a patient's body captured from different angles and distances into a digital 3D image of the portion. In an example, machine learning can be used to integrate images of a portion of a patient's body captured from different angles and distances into a digital 3D image of the portion.

In an example, deep learning algorithms can be used to integrate images of a portion of a patient's body captured from different angles and distances into a digital 3D image of the portion. In an example, fuzzy logic can be used to integrate images of a portion of a patient's body captured from different angles and distances into a digital 3D image of the portion. In an example, an artificial neural network can be used to integrate images of a portion of a patient's body captured from different angles and distances into a digital 3D image of the portion. In an example, Fourier transformation can be used to integrate images of a portion of a patient's body captured from different angles and distances into a digital 3D image of the portion.

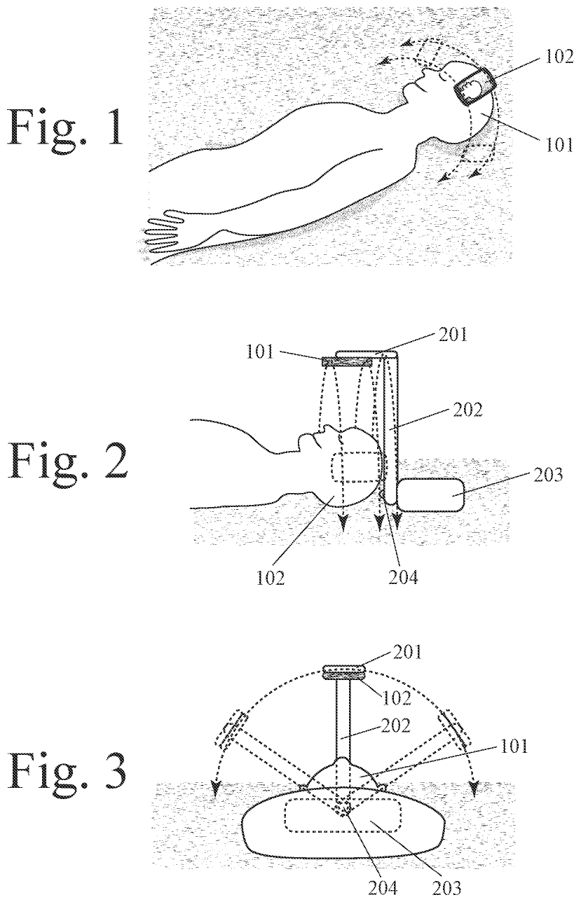

FIGS. 1 through 15 are now described. Example variations discussed in the prior section can be applied to these examples where relevant.

FIG. 1. shows an example of how a cellphone 102 can be moved in an arcuate path in space over (e.g. over, above, across, and/or around) a portion of a patient's body 101 in order to capture images of the portion from different angles and distances. FIG. 1 shows this movement from an oblique side perspective. In this example, the arcuate path is a semicircular path. In this example, a cellphone is moved over e.g. over, above, across, and/or around) a patient's head. In this example, a cellphone is moved in a semicircular path over (e.g. over, above, across, and/or around) the head of a patient who is lying down. Images of the portion of the patient's body captured by the cellphone from different angles and distances as the cellphone is moved along the arcuate path can be integrated into a digital 3D image. The digital 3D image can be: analyzed by machine learning methods and/or artificial intelligence; and/or examined by a remote healthcare provider.

In an example, a cellphone can be moved in an arcuate path in space by a person, wherein the person receives real-time auditory, visual, or tactile guidance from machine learning methods and/or artificial intelligence concerning how to move the cellphone. In an example, auditory guidance can be via machine-generated vocal directions or sounds. In an example, visual guidance can be via visual directions or cues shown in an augmented reality display. In another example, a cellphone can be moved in an arcuate path in space by a specialized cellphone-moving device. In an example, a cellphone can be removably-attached to a moving arm of such a cellphone-moving device. In another example, a cellphone can be moved in an arcuate path by a general-purpose home robot.

FIGS. 2 and 3 show two views of an example of specialized-cellphone moving device which moves a cellphone in an arcuate path in space over (e.g. over, above, across, and/or around) a portion of a patient's body in order to capture images of the portion from different angles and distances. FIG. 2 shows this device from a first side perspective. FIG. 3 shows this device from a second side perspective

With respect to specific components, FIGS. 2 and 3 show a cellphone-moving device comprising: a base 203; a pivoting arm 202 which is attached to the base by an axle 204; and a cellphone dock 201 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion 102 of a patient's body in order to capture images of the portion from different angles and distances.

In this example, the arcuate path is a semicircular. In this example, a portion of a patient's body is their head. In this example, a cellphone is moved in a semicircular path over (e.g. over, above, across, and/or around) the head of a patient who is lying down. Images of the portion of the patient's body captured by the cellphone from different angles and distances as the cellphone is moved along the arcuate path can be integrated into a digital 3D image for: analysis by machine learning methods and/or artificial intelligence; and/or examination by a remote healthcare provider.

In this example, the pivoting arm is moved back and forth like a windshield wiper or the arm in a metronome. In this example, a location on the lower half of the pivoting arm is attached to the base and the cellphone dock is attached to a location on the upper half of the pivoting arm. In an example, the device can further comprise an electromagnetic motor which moves the pivoting arm. In this example, the cellphone dock extends out in a perpendicular manner from the pivoting arm. In an example, the base can be weighted so that the cellphone dock can extend out from the pivoting arm without tipping over the device. Example variations discussed elsewhere in this disclosure or in priority-linked disclosures can be applied to this example where relevant.

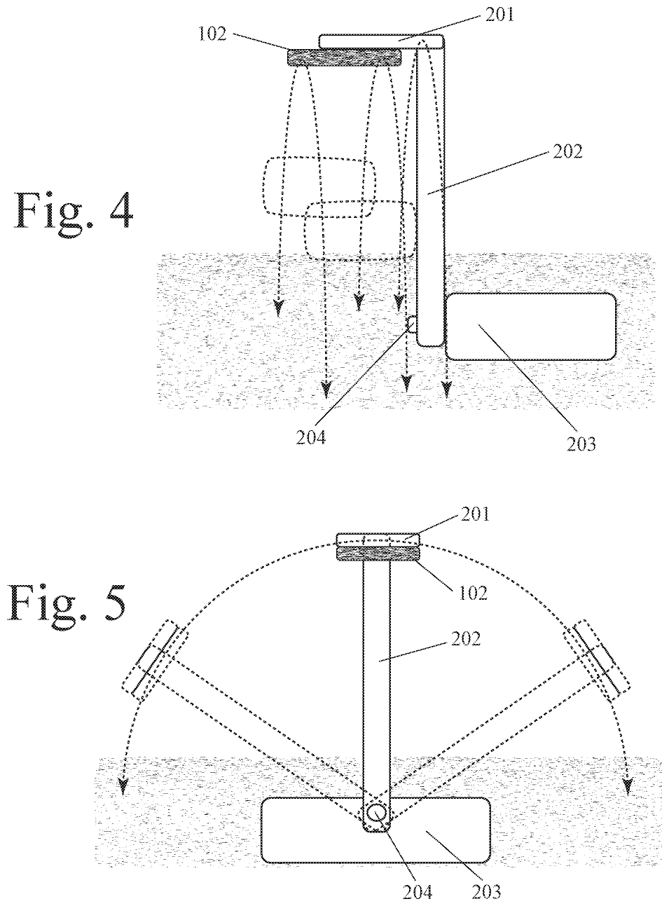

FIGS. 4 and 5 show two close-up views of the example of specialized-cellphone moving device which was introduced in FIGS. 2 and 3, without showing the body of the patient. Showing the device without the body of the patient enables a less-cluttered view of the device alone. FIG. 4 shows this device from a first side perspective. FIG. 5 shows this device from a second side perspective.

With respect to specific components, FIGS. 4 and 5 show a cellphone-moving device comprising: a base 203; a pivoting arm 202 which is attached to the base by an axle 204; and a cellphone dock 201 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion of a patient's body in order to capture images of the portion from different angles and distances.

In this example, the arcuate path is semicircular. Images of the portion of the patient's body captured by the cellphone from different angles and distances as the cellphone is moved along the arcuate path can be integrated into a digital 3D image for: analysis by machine learning methods and/or artificial intelligence; and/or examination by a remote healthcare provider.

In this example, the pivoting arm is moved back and forth like a windshield wiper or the arm in a metronome. In this example, a location on the lower half of the pivoting arm is attached to the base and the cellphone dock is attached to a location on the upper half of the pivoting arm. In an example, the device can further comprise an electromagnetic motor which moves the pivoting arm. In this example, the cellphone dock extends out in a perpendicular manner from the pivoting arm. In an example, the base can be weighted so that the cellphone dock can extend out from the pivoting arm without tipping over the device. Example variations discussed elsewhere in this disclosure or in priority-linked disclosures can be applied to this example where relevant.

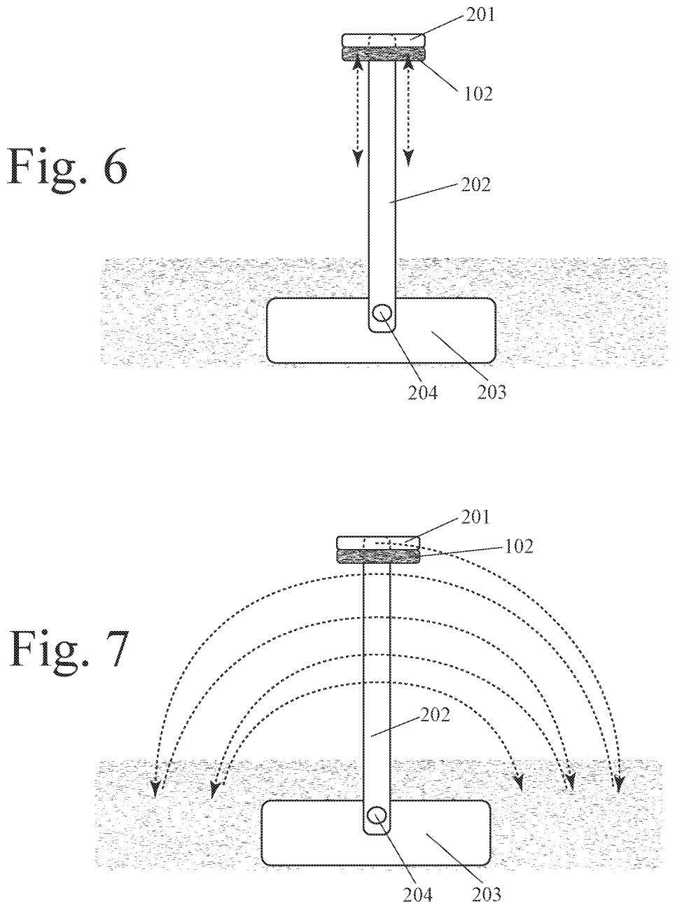

FIG. 6 shows an example of how the length of an arm of a cellphone-moving device can be adjusted. With respect to specific components, FIG. 6 shows a cellphone-moving device comprising: a base 203; a pivoting arm 202 which is attached to the base by an axle 204, wherein the length of the arm is adjustable; and a cellphone dock 201 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion of a patient's body in order to capture images of the portion from different angles and distances. In an example, instead of adjusting the full length of the arm, the cellphone dock can be moved along (e.g. slid up or down on) the arm to change size of the arcuate path. Example variations discussed elsewhere in this disclosure or in priority-linked disclosures can be applied to this example where relevant.

FIG. 7 shows an example of how adjustment of the length of a pivoting arm on a cellphone-moving device can interact with back and forth movement of the arm. In this example, decreasing the length of the pivoting arm as it pivots back and forth causes the cellphone to move in a serpentine oscillating pattern. This helps to capture images of the portion of the person's body from different angles and distances. These images are then integrated to create a digital 3D image of the portion of the person's body.

With respect to specific components, FIG. 7 shows a cellphone-moving device comprising: a base 203; a pivoting arm 202 which is attached to the base by an axle 204, wherein the length of the arm is adjustable; and a cellphone dock 201 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion of a patient's body in order to capture images of the portion from different angles and distances. In an example, instead of adjusting the full length of the arm as the arm pivots, the cellphone dock can be moved along (e.g. slid up or down on) the arm as the arm pivots to create the serpentine oscillating pattern. Example variations discussed elsewhere in this disclosure or in priority-linked disclosures can be applied to this example where relevant.

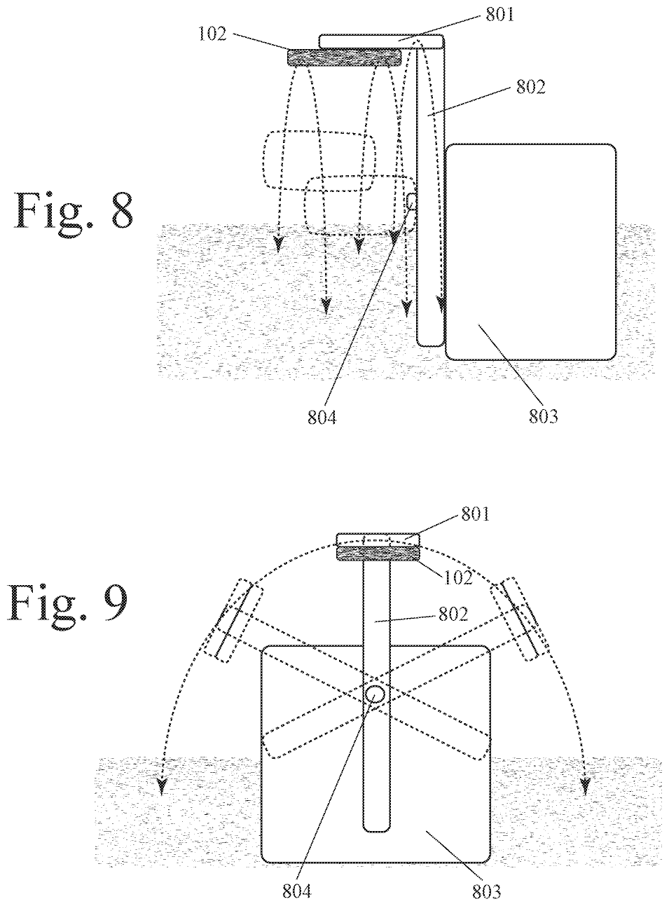

FIGS. 8 and 9 show two views of an example of specialized-cellphone moving device which moves a cellphone in an arcuate path in space over (e.g. over, above, across, and/or around) a portion of a patient's body in order to capture images of the portion from different angles and distances. FIG. 8 shows this device from a first side perspective. FIG. 9 shows this device from a second side perspective. This device is similar to the one shown in FIGS. 2 and 3 except that the arm is connected to the base at a central location.

With respect to specific components, FIGS. 8 and 9 shows a cellphone-moving device comprising: a base 803; a pivoting arm 802 which is centrally-attached to the base by an axle 804; and a cellphone dock 201 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion of a patient's body in order to capture images of the portion from different angles and distances.

In this example, the arcuate path is a semicircular. In this example, a location on a central portion (e.g. the central third) of the pivoting arm is attached to the base and the cellphone dock is attached to a location on the upper half of the pivoting arm. In an example, the device can further comprise an electromagnetic motor which moves the pivoting arm. In this example, the cellphone dock extends out in a perpendicular manner from the pivoting arm. In an example, the base can be weighted so that the cellphone dock can extend out from the pivoting arm without tipping over the device. Example variations discussed elsewhere in this disclosure or in priority-linked disclosures can be applied to this example where relevant.

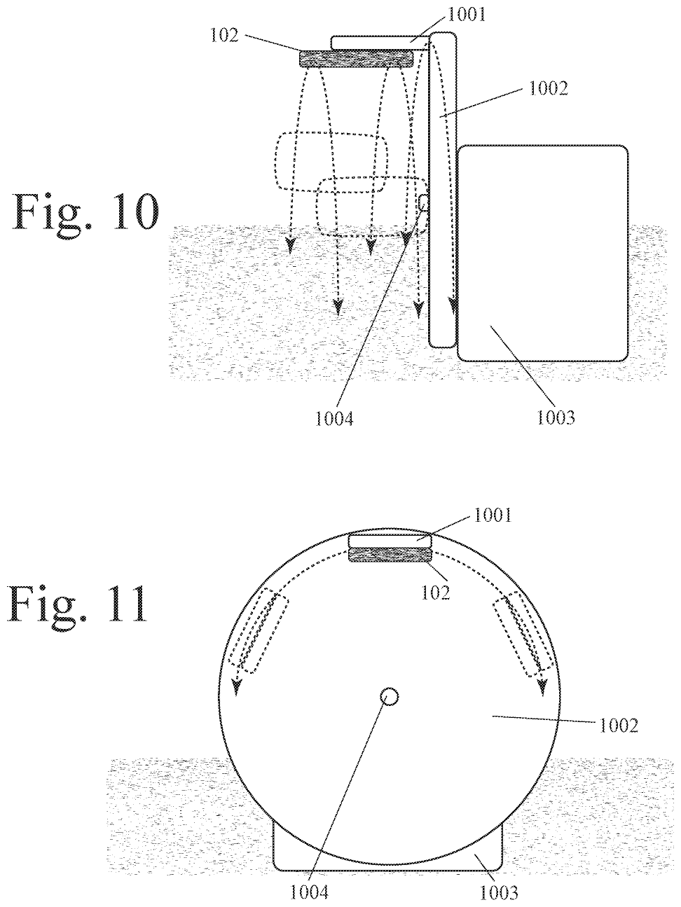

FIGS. 10 and 11 show two views of an example of specialized-cellphone moving device which moves a cellphone in an arcuate path in space over (e.g. over, above, across, and/or around) a portion of a patient's body in order to capture images of the portion from different angles and distances. FIG. 10 shows this device from a first side perspective. FIG. 11 shows this device from a second side perspective. This device is similar to the one shown in FIGS. 2 and 3 except that the device moves the cellphone with a revolving wheel instead of a pivoting arm.

With respect to specific components, FIGS. 10 and 11 show a cellphone-moving device comprising: a base 1003; a revolving wheel 1002 which is attached to the base by an axle 1004; and a cellphone dock 1001 which is part of (or attached to) the pivoting arm, wherein a cellphone 101 is removably-attached to the cellphone dock, and wherein the cellphone is moved by the arm in an arcuate path in space over (e.g. over, above, across, and/or around) a portion of a patient's body in order to capture images of the portion from different angles and distances.

In this example, the arcuate path is a semicircular. In an example, the device can further comprise an electromagnetic motor which revolves the wheel. In this example, the cellphone dock extends out in a perpendicular manner from the wheel. In an example, the base can be weighted so that the cellphone dock can extend out from the wheel without tipping over the device. Example variations discussed elsewhere in this disclosure or in priority-linked disclosures can be applied to this example where relevant.

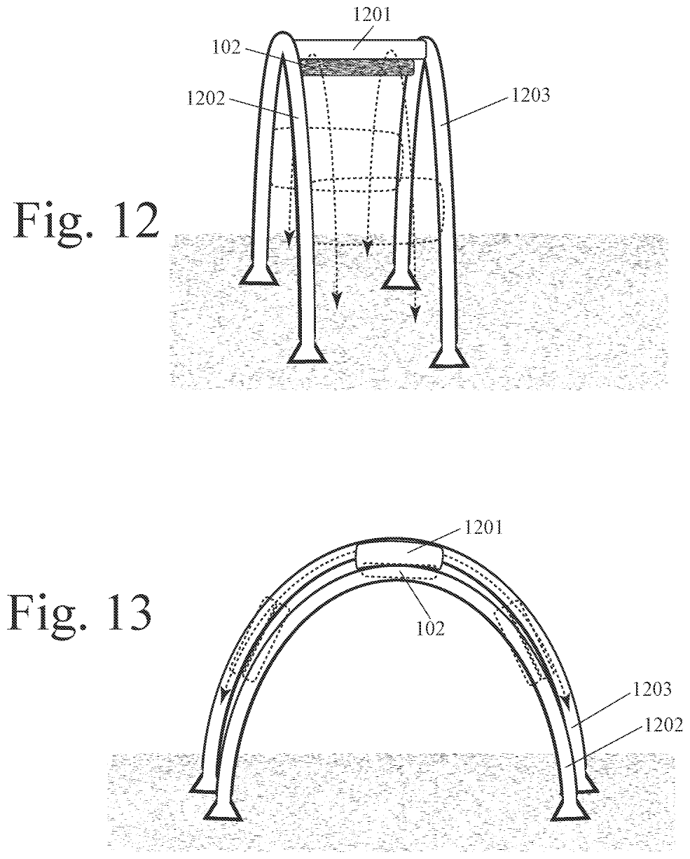

FIGS. 12 and 13 show two views of an example of a cellphone moving device with two arches which moves a cellphone in an arcuate path in space over (e.g. over, above, across, and/or around) a portion of a patient's body in order to capture images of the portion from different angles and distances. FIG. 12 shows this device from a first side perspective. FIG. 13 shows this device from a second side perspective.

With respect to specific components, FIGS. 12 and 13 show a cellphone-moving device comprising: two arches 1202 and 1203; and a cellphone dock 1201 which is moveably-attached the two arches, wherein a cellphone 101 is removably-attached to the cellphone dock, and wherein the cellphone is moved along the two arches in an arcuate path in space over (e.g. over, above, across, and/or around) a portion of a patient's body in order to capture images of the portion from different angles and distances.

In an example, first ends of the two arches can be placed to the right of a portion of a patient's body and second ends of the two arches can be placed to the left of the portion person's body, wherein the arches span over (e.g. over, above, across, and/or around) the portion of the patient's body. In an example, the ends of the arches can have expanded sections (e.g. feet) to help keep them stable. In an example, the two arches can be connected to each other at their ends to help keep them stable. In an example, the arches can have optional connectible sections which can be inserted to increase their arcuate span for imaging a larger portion of a patient's body. In an example, the arches can be shipped in sections to a patient's home and then assembled at a patient's home

In an example, the cellphone dock can be between the two arches. In an example, the cellphone dock can move along the two arches in an arcuate path in space. In an example, the cellphone dock can be moved along the arches by rotating wheels on the dock which frictionally-engage the arches. In an example, the cellphone dock can be moved along the arches by gears on the dock which engage cogs or tracks on the arches. In an example, the cellphone dock can be moved along the arches by a moving belt, chain, wire, or cord which moves through the arches. Example variations discussed elsewhere in this disclosure or in priority-linked disclosures can be applied to this example where relevant.

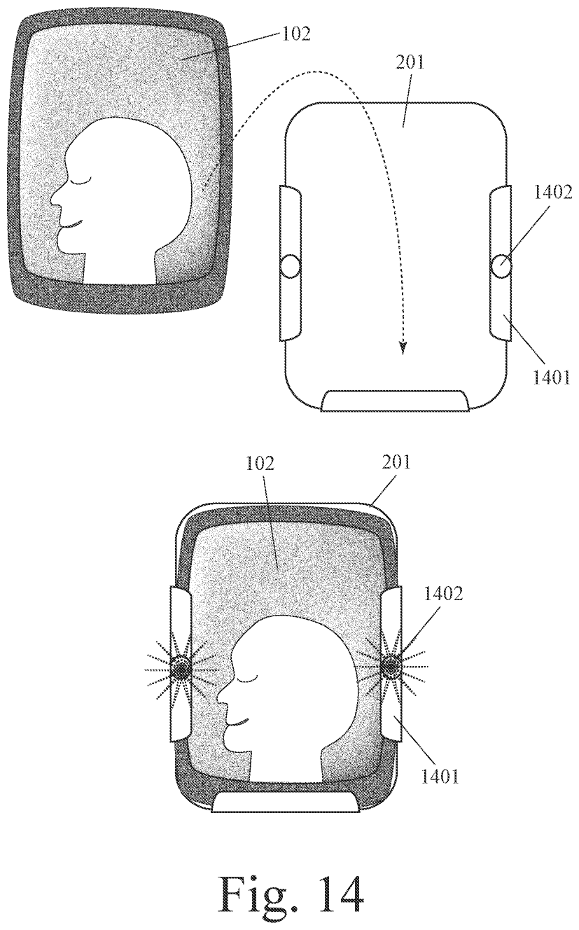

FIG. 14 shows a close-up view of a cellphone dock and how a cellphone can be removably-attached to the dock. The upper portion of FIG. 14 shows a cellphone and a cellphone dock when they are separate. The lower portion of FIG. 14 shows them after the cellphone has been removably-attached to the cellphone dock. With respect to specific components, FIG. 14 shows a cellphone 102 which is slid into tracks (e.g. tracks, grooves, clamps, or clips), including track 1401, on a cellphone dock 201. In this example, the cellphone dock also includes one or more light emitters, including light emitter 1402.

In this example, the cellphone slides into tracks (e.g. tracks, grooves, clamps, or clips) on the cellphone dock. In this example, there are three tracks on the cellphone, one on each side of the dock and one at one end of the cellphone, which hold the cellphone onto the cellphone dock. The dock is part of a specialized cellphone-moving device, the rest of which is not shown in this figure. In this example, the cellphone dock includes one or more light emitters (e.g. LEDs). In this example, there are two light emitters on each side of the cellphone dock. Example variations discussed elsewhere in this disclosure or in priority-linked disclosures can be applied to this example where relevant.

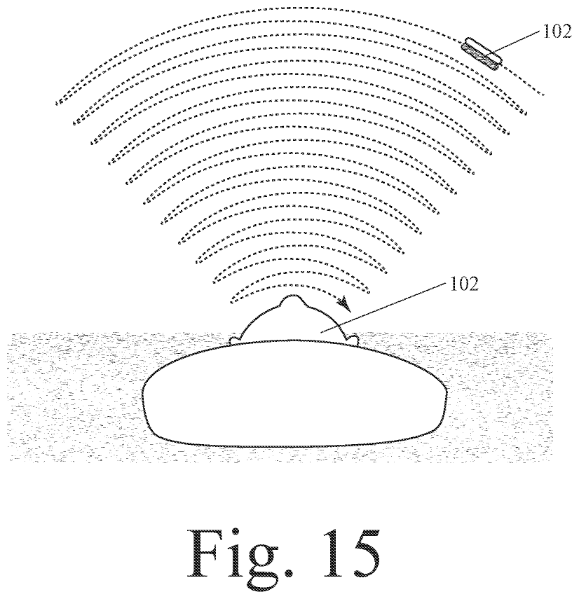

FIG. 15 shows an example of how movement of a cellphone 102 along an arcuate path in space above a portion of a patient's body 102 can capture images of the portion from different angles and distances. In this example, the distance between the cellphone and the portion of the patient's body is decreased as the cellphone is moved back and forth along arcs over the portion of the patient's body. The resulting arcuate path is a serpentine oscillating path with an overall perimeter which is shaped like a paper fan, a keystone, or an area on a windshield which is wiped by a windshield wiper. Example variations discussed elsewhere in this disclosure or in priority-linked disclosures can be applied to this example where relevant.

I claim:

1. A circumferential array of electromyographic sensors comprising:
a plurality of circumferential rings of electromyographic sensors which are configured to encircle a selected body member of a person; wherein the selected body member is selected from the group consisting of the person's arm, wrist, hand, finger, leg, ankle, or foot; wherein electromyographic sensors in a ring of electromyographic sensors are at different polar coordinate locations around a circumference of the selected body member; and wherein distances between the electromyographic sensors are adjusted by an electromagnetic actuator, an inflatable chamber or pneumatic member, a hydraulic mechanism, or an adjustable spring; and
a plurality of columns of electromyographic sensors which are configured to be substantially parallel to a longitudinal axis of the selected body member.

2. The circumferential array in claim 1 wherein the polar coordinate locations of electromyographic sensors are evenly spaced around the circumference of the selected body member.

3. The circumferential array in claim 1 wherein the circumferential array is incorporated into an arm band, bangle, or bracelet.

4. The circumferential array in claim 1 wherein the circumferential array is incorporated into a wrist band.

5. The circumferential array in claim 1 wherein the circumferential array is incorporated into a watch strap.

6. The circumferential array in claim 1 wherein the circumferential array is incorporated into the cuff or sleeve of an article of clothing.

7. The circumferential array in claim 1 wherein different sensing roles are assigned to different electromyographic sensors around the circumference of the selected body member in order to correct for physical shifting or rotation of these electromyographic sensors around the selected body member.

8. The circumferential array in claim 1 wherein a ring of electromyographic sensors spans in the range of 50% to 75% of the circumference of a cross-section of the selected body member.

9. The circumferential array in claim 1 wherein a ring of electromyographic sensors spans in the range of 75% to 100% of the circumference of a cross-section of the selected body member.

10. A circumferential array of electromyographic sensors comprising:
at least three circumferential rings of electromyographic sensors which are configured to encircle a selected body member of a person; wherein the selected body member is selected from the group consisting of the person's arm, wrist, hand, finger, leg, ankle, or foot; wherein electromyographic sensors in a ring of electromyographic sensors are at different polar coordinate locations around a circumference of the selected body member; and wherein the electromyographic sensors are configured to be compelled radially inward toward the body member by an electromagnetic actuator, an inflatable chamber or pneumatic member, a hydraulic mechanism, or an adjustable spring; and
at least three of columns of electromyographic sensors which are configured to be substantially parallel to a longitudinal axis of the selected body member.

11. The circumferential array in claim 10 wherein the polar coordinate locations of electromyographic sensors are evenly spaced around the circumference of the selected body member.

12. The circumferential array in claim 10 wherein the circumferential array is incorporated into an arm band, bangle, or bracelet.

13. The circumferential array in claim 10 wherein the circumferential array is incorporated into a wrist band or watch strap.

14. The circumferential array in claim 10 wherein the circumferential array is incorporated into the cuff or sleeve of an article of clothing.

15. The circumferential array in claim 10 wherein different sensing roles are assigned to different electromyographic sensors around the circumference of the selected body member in order to correct for physical shifting or rotation of these electromyographic sensors around the selected body member.

16. The circumferential array in claim 10 wherein a ring of electromyographic sensors spans in the range of 50% to 75% of the circumference of a cross-section of the selected body member.

17. The circumferential array in claim 10 wherein a ring of electromyographic sensors spans in the range of 75% to 100% of the circumference of a cross-section of the selected body member.

18. (canceled)
